(19) United States
(12) Patent Application Publication
(10) Pub. No.: US 2019/0022127 A1
(43) Pub. Date: Jan. 24, 2019

(54) LONG NON-CODING RNA LncHIFCAR/MIR31HG AND ITS APPLICATIONS

(71) Applicants: TAIPEI MEDICAL UNIVERSITY, Taipei City (TW); CHI MEI MEDICAL CENTER, Tainan city (TW); NATIONAL HEALTH RESEARCH INSTITUTES, Miaoli County (TW)

(72) Inventors: Hsing-Jien KUNG, Sacramento, CA (US); Wei-Fan Chiang, Tainan (TW); Jing-Wen Shih, Taichung City (TW)

(21) Appl. No.: 16/041,111

(22) Filed: Jul. 20, 2018

Related U.S. Application Data
(60) Provisional application No. 62/534,901, filed on Jul. 20, 2017.

(51) Int. Cl. A61K 31/7105; C12Q 1/6886; A61P 35/00

(52) U.S. Cl. CPC A61K 31/7105; A61P 35/00; C12Q 1/6886

(57) ABSTRACT

The invention is related to LncHIFCAR (long noncoding HIF-1α co-activating RNA)/MIR31HG and its applications in cancer diagnosis, cancer therapy, prognosis predication of a cancer and determination of therapeutic regimen of a cancer. The present invention identifies a hypoxia-inducible lncRNA, LncHIFCAR (long noncoding HIF-1α co-activating RNA)/MIR31HG, and describes its oncogenic role as a HIF-1α co-activator that regulates the HIF-1 transcriptional network, crucial for cancer development.

### FIELD OF THE INVENTION

The present invention is related to the fields of cancer diagnosis, cancer therapy, prognosis predication of a cancer and determination of therapeutic regimen of a cancer. Particularly, the invention is related to LncHIFCAR (long noncoding HIF-1α co-activating RNA)/MIR31HG and its applications in cancer diagnosis, cancer therapy, prognosis predication of a cancer and determination of therapeutic regimen of a cancer.

### BACKGROUND OF THE INVENTION

Hypoxia is a common feature of rapidly growing solid tumors, tightly associated with tumor metastasis and poor prognosis, and a contributor to malignant progression and aggressive phenotype in many cancer types. Hypoxia-inducible factor-1 (HIF-1), a heterodimer consisting of α and β subunits, is a key regulator of the cellular response to hypoxia. Under hypoxic conditions, the HIF-1α subunit is stabilized and translocated to the nucleus where it forms a stable HIF-1 complex, specifically bound to the promoter regions of HIF-1 target genes and thereby inducing gene transcription. Proteins encoded by HIF-1 target genes are involved in multiple aspects of tumorigenesis, including glucose and energy metabolism, proliferation, cancer stem-like properties, angiogenesis, invasion and metastasis. The activation of HIF-1 pathways is associated with an aggressive tumor phenotype and poor clinical outcome in numerous cancer types, including oral cancer. For example, oral squamous cell carcinoma (OSCC) represents one of the most common malignancies worldwide with a high mortality rate mainly due to lack of early detection markers, frequent association with metastasis and aggressive phenotype.

Thus, there is a need to identify biomarkers and therapeutic targets for a cancer.

### SUMMARY OF THE INVENTION

The present invention identifies a hypoxia-inducible lncRNA, LncHIFCAR (long noncoding HIF-1α co-activating RNA)/MIR31HG, and describes its oncogenic role as a HIF-1α co-activator that regulates the HIF-1 transcriptional network, crucial for cancer development. Extensive analyses of clinical data indicate LncHIFCAR level is substantially up-regulated in carcinoma (particularly, oral carcinoma), significantly associated with poor clinical outcomes and representing an independent prognostic predictor. Overexpression of LncHIFCAR induces pseudo-hypoxic gene signature, whereas knockdown of LncHIFCAR impairs the hypoxia-induced HIF-1α transactivation, sphere-forming ability, metabolic shift and metastatic potential in vitro and in vivo. Mechanistically, LncHIFCAR forms a complex with HIF-1α via direct binding, and facilitates the recruitment of HIF-1α and p300 cofactor to the target promoters. The invention uncovers a lncRNA-mediated mechanism for HIF-1 activation, and establishes the values of LncHIFCAR in diagnosis, prognosis and potential therapeutic strategy for carcinoma.

In one aspect, the invention provides a method of diagnosing whether a subject has, or is at risk for a cancer, a metastatic cancer or a primary cancer, comprising: (a) isolating a LncHIFCAR transcript in a biological sample from the subject; (b) measuring a test level of the isolated LncHIFCAR transcript; (c) comparing the test level to a control level of the LncHIFCAR transcript; and (d) determining a subject as having the cancer, metastatic cancer or primary cancer when the test level is higher than the control level.

In another aspect, the invention provides a method of determining a prognosis, recurrence-free survival or overall survival of a subject having, or suspected of a cancer, a metastatic cancer or a primary cancer, comprising: a) isolating a LncHIFCAR transcript in a biological sample from the subject; b) measuring a test level of the isolated LncHIFCAR transcript; c) comparing the test level to a control level of the LncHIFCAR transcript; and d) determining a subject as having a poor prognosis, poor recurrence-free survival or poor overall survival when the test level is higher than the control level.

In another aspect, the invention provides a kit for predicting a risk for developing an oral cancer, a metastatic oral cancer or a primary oral cancer or a prognosis, recurrence-free survival or overall survival of a subject, comprising reagents for determining a level of the LncHIFCAR in the sample.

In a further another aspect, the invention provides a method of treating an oral cancer, a metastatic oral cancer or a primary oral cancer in a subject comprising administering to the subject an effective amount of a therapeutic agent that blocks an expression or overexpression of MIR31HG gene or a physiological action of a LncHIFCAR transcript.

### BRIEF DESCRIPTION OF THE DRAWING

FIG. 1 shows that LncHIFCAR expression is induced upon hypoxia and highly up-regulated in human oral carcinoma with prognostic value. (a,b) The expression profiling of 37 cancer-associated lncRNAs in HeLa cells under hypoxia (1% O₂ for 16 hours; a) or treated with hypoxia-mimetic agent cobalt chloride (100 μM for 16 hours; b) was assessed by quantitative real time PCR and normalized against 18S rRNA. Each bar represents the relative expression of each lncRNA after treatment. The Venn diagram shows the number of lncRNAs that were up-regulated in response to hypoxia or cobalt chloride treatment. (c) Physical hypoxia induced the expression of LncHIFCAR in a time-dependent manner within 48 hours. HeLa cells were incubated in 1% oxygen for the indicated time period. Real-time PCR analyses determining the expression levels of LncHIFCAR and the intrinsic hypoxia marker CA9 mRNA were shown and normalized to 18S rRNA level. (d) LncHIFCAR expression in OSCC patient samples analyzed in Peng Head-Neck dataset (GEO: GSE25099) from Oncomine database. The LncHIFCAR expression is presented as box-plot diagrams, with the box encompassing 25th-75th percentile. Solid horizontal black line represents the median while error bars indicate the 10th to 90th percentile. Statistical analyses between different patient groups were examined by Student's t test, ***P<0.001. (e) LncHIFCAR expression in 15 pairs of OSCC tissues (Tumor) and their normal counterpart tissues (Normal) was analyzed by quantitative real-time PCR, and normalized to RPLP0. The LncHIFCAR expression showing more than 2-fold change between normal and tumor samples were considered significant. (f,g) Kaplan-Meier curves of overall (f) and recurrence-free survival (g). Patients were grouped into LncHIFCAR-Low or LncHIFCAR-High based on the LncHIFCAR expression value. The cutoff point is set as the value yielding maximum sum of sensitivity and specificity for recurrence-free survival analysis. The P-values was determined by log-rank test, *P<0.05; **P<0.01. (c,e) Graphs show mean±SD. n, the number of independent experiments. (h,i and j). Patients with high LncHIFCAR expression level had a significantly worse uterine corpus endometrial carcinoma, glioblastoma and kidney renal cell carcinoma overall survival than those with low LncHIFCAR expression.

FIG. 2 shows that LncHIFCAR-knockdown impairs hypoxia-enhanced migration/invasion capability, glycolytic metabolism and tumor sphere formation in human oral cancer SAS cells. (a) Expression levels of LncHIFCAR in oral cancer cell lines. The levels of LncHIFCAR were normalized against RPLP0 mRNA level and are presented relative to the expression in OEC-M1. (b) Establishment of LncHIFCAR-knockdown SAS clones. Upper panel: schematic representation of the genomic structure of LncHIFCAR/MIR31HG. The targeting sites of the designed siRNAs are indicated by gray arrowhead. LncHIFCAR/MIR31HG is composed of four exons (filled blue boxes), and the miR-31, located within intron 1 is shown with a red horizontal bar. Lower panel: LncHIFCAR and miR-31 expression levels in siRNA-transfected SAS cells were analyzed by quantitative PCR and normalized to GAPDH and U6 level, respectively. (c) LncHIFCAR promotes oral cancer cell invasion and migration. Transwell invasion and migration assays of siRNA-transfected SAS cells were performed. Representative photos and quantitative analysis are shown. (Scale bar, 100 μm) (d) Cell growth curve of the siRNA-transfected SAS cells under normoxia or hypoxia. (e,f) Knockdown of LncHIFCAR reduces hypoxia-induced glucose uptake (e) and lactate production (f). The glucose and lactate levels in the culture media of the siRNA-transfected SAS cells were measured after 16 hours of normoxia or hypoxia treatment. The data is presented as fold difference compared with the level of negative control siRNA-transfected cells in normoxia. (g) Knockdown of LncHIFCAR reduces the sphere forming ability of SAS cells. The siRNA-transfected SAS cells were grown in suspension culture to form sphere. Representative phase-contrast microscopic images of the cell aggregates are displayed (Scale bar, 300 μm). Bar graph represents the total number of spheres or the relative number of spheres with diameter >1 mm. Graphs show mean±SD. NC, negative control. n, the number of independent experiments performed; Student's t test, *P<0.05; **P<0.01; ***P<0.001.

FIG. 3 shows that LncHIFCAR is required for the hypoxia-induced activation of HIF-1 target genes and the HIF-1-dependent transactivation. (a) The RNA levels of HIF1A and HIF-1 target genes in HeLa cells transfected with vector, LncHIFCAR- or RMRP-expressing plasmids for 48 hours measured by qRT-PCR. (b) The RNA levels of HIF-1 target genes in HeLa cells transfected with different amount of the LncHIFCAR-expressing plasmid measured by qRT-PCR. (c) HIF-1 target genes expression in HeLa cells transfected with the indicated siRNAs. 24 hours after transfection, the cells were treated by CoCl₂ for 16 hours, followed by qRT-PCR analysis with normalization against RPLP0 level. (d) qRT-PCR analysis of HIF-1 target genes expression (normalized to RPLP0) in siRNA-transfected SAS cells under 24 hours treatment of normoxia or hypoxia. (e) The expression of LncHIFCAR and HIF-1 target genes in tumor spheres derived from HSC3 and SAS cells (normalized to GAPDH), compared with monolayer cultures of the parental cells. Insets, representative phase contrast microscopic images of the monolayer and sphere cells (Scale bar, 200 μm). The 18S rRNA level is served as a control. (f) Developing hypoxic region at the core of the spheres derived from SAS oral cancer cells was observed in immunostaining preparations using an exogenous hypoxia marker pimonidazole (Scale bar, 100 μm). (g) The expressional correlation between LncHIFCAR and HIF-1 target genes (LDHA and L1CAM) in the HNSCC (TCGA, Provisional) study (n=522) were surveyed using cBioPortal platform. The corresponding correlation plots are shown with Pearson coefficients. (h,i) HIF-1α responsive luciferase reporter assays in LncHIFCAR overexpressing (h) or LncHIFCAR knockdown (i) cells presented as relative value with normalization against Renilla-Luc activity (h) A plasmid containing HIF-1α responsive luciferase reporter was co-transfected with empty vector, HIF-1a, or LncHIFCAR into HeLa cells for the reporter assay. (i) Reporter assay performed in siRNA-transfected HeLa cells co-transfected with the indicated plasmids for 24 hours, followed by 24-hour treatment of hypoxia or normoxia. Graphs show mean±SD. NC, negative control. n, the number of independent experiments performed; Student's t test, *P<0.05; **P<0.01; ***P<0.001.

FIG. 4 shows that lncRNA LncHIFCAR associates with HIF-1α and functions as a HIF-1α co-activator. (a) Representative (n=3) western blot analysis of HIF-1α protein levels in LncHIFCAR-overexpressing HeLa cells and controls (lower panel), or in siRNA-transfected SAS cells (upper panel) under 24 hours of normoxia (N) or hypoxia (H) treatment. NC, negative control. (b) lncRNA LncHIFCAR enriched in HIF-1α immunoprecipitates under hypoxia. 16-hour normoxia- or hypoxia-treated SAS nuclear extracts were immunoprecipated using mouse IgG or anti-HIF-1α antibody. Immunoprecipitation of HIF-1α-associated RNA was validated by qRT-PCR and shown as the relative fold of the RNA enrichment. (c) Representative (n=3) immunoblot detection of HIF-1α retrieved by biotinylated LncHIFCAR RNA pull-down. Biotin-labeled lambda RNA, sense or antisense LncHIFCAR RNAs were incubated with 16-hour hypoxia-treated HeLa nuclear extracts, and then pulled down by streptavidin beads, followed by western blot analysis. (d,e) HIF-1α binding domain on LncHIFCAR. RNAs corresponding to indicated LncHIFCAR fragments were biotinylated and incubated with recombinant HIF-1α protein, followed by streptavidin pull-down as described above. Representative (n=3) immunoblot detection of the associated HIF-1α protein was shown. CB, Coomassie brilliant blue staining. (f) HIF-1 target gene expression in SAS cells overexpressing control vector, wild-type or mutant (5000-1500) LncHIFCAR RNA. Specific primers are designed for the quantification of wild-type (A), or mutant (5000-1500) (B) LncHIFCAR levels as shown in the schematic diagram, and the primer sequences are provided in Supplementary Data 2. (g) LncHIFCAR binding domain within HIF-1α. Schematic representation of HIF-1α functional domains and GST-HIF-1α variants are shown at the top. The Coomassie Blue staining showed loading of the proteins and arrowheads mark the GST-HIF-1α truncates. LncHIFCAR RNA were pulled down by GST-fusion proteins pre-bound on glutathione-Sepharose beads, followed by qRT-PCR detection of LncHIFCAR RNA retrieved as presented as percentage relative to input RNA. bHLH, basic helix-loop-helix; PAS, Per-ARNT-Sim; TAD, transactivation domain. (h) LncHIFCAR facilitates HIF-1α complex formation. HIF-1α was immunoprecipitated from hypoxic vector control or LncHIFCAR knockdown SAS cell extracts. Co-immunoprecipitation of p300 and HIF-1β was reduced in LncHIFCAR knockdown cells. Graphs show mean±SD. n, the number of independent experiments performed.

FIG. 5 shows that LncHIFCAR physically binds to the target chromatin and enhances the recruitment of HIF-1α and p300. (a) ChIRP-qPCR detection of LncHIFCAR occupancy on the indicated target loci under normoxic and hypoxic conditions. ChIRP assays were performed with hypoxia (H) or normoxia (N) treated SAS cell lysate. Specific tiling biotinylated oligonucleotides complementary to LncHIFCAR or control LacZ RNA were used to pull down the RNA-associated chromatin. The Inset graph shows the retrieved RNA level in the streptavidin-pulled down complex, quantified by qRT-PCR. GAPDH mRNA was used to evaluate nonspecific binding of the biotinylated probes. The LncHIFCAR-associated HIF-1 target promoters were detected by qPCR. The retrieval of DNA was estimated as percentage of input chromatin while Actin promoter served as a negative control region. (b,c) ChIP analysis of HIF-1α (b) and p300 (c) association with the promoter regions of HIF-1 target genes. The chromatin was prepared from vector control or LncHIFCAR knockdown SAS cell lines treated with normoxia (N) or hypoxia (H). ChIP was performed with anti-HIF-1α, anti-p300, or IgG and the recovered DNA was analyzed by qPCR. The fold enrichment, indicated by fold of IgG, was calculated by normalizing the levels against nonspecific IgG-bound DNA. Inset, protein levels of HIF-1α and p300 in control and LncHIFCAR knockdown cells under normoxia (N) and hypoxia (H). Graphs show mean±SD. n, the number of independent experiments performed.

FIG. 6 shows that LncHIFCAR is an oncogenic driver of metastatic cascade. SAS cells stably expressing control (sh-Ctrl) or LncHIFCAR knockdown (sh-HIFCAR) construct with firefly luciferase labeling were injected into the tail vein of nu/nu mice (n=10 each group). The metastatic tumor nodules in the lungs of the animals were monitored weekly by luciferin injection and IVIS imaging. (a,b) The bioluminescence intensity of the lung area (a) and total signal (b) of the animals were quantified at the indicated time points. (c) Representative bioluminescence images of 5 mice in each group at day 42 after tail vein injections. (d,e) Representative images of the metastatic lung nodules (d; scale bar, 5 mm) and haematoxylin and eosin (H&E) staining (e; scale bar, 500 μm) of the lung sections from mice in each group 6 weeks after tail vein injections. The black arrowheads indicate the metastatic nodules. (f) Quantitation of the number of pulmonary nodules in each mice group 6 weeks after tail vein injection. (g) A proposed model that depicts how lncRNA LncHIFCAR acts as a HIF-1α co-activator via its interaction with HIF-1α that contributes to the activation of HIF-1 transcriptional network associating with cancer progression. The possible utility of elevated LncHIFCAR expression as a prognostic biomarker and therapeutic target for OSCC is indicated. Graphs show mean±SD. Student's t-test, *P<0.05, **P<0.01, ***P<0.001).

### DETAILED DESCRIPTION OF THE INVENTION

The invention found MIR3HG as a species that is significantly up-regulated in cancers. The invention describes a previously unrecognized role of the mature, spliced form of MIR31HG as a co-activator of HIF-1α that activates the pseudohypoxia signature required for hypoxia-induced metabolic reprogramming, sphere-forming ability and metastatic potential. As this RNA species does not contain miR-31 sequence and functions independently, it is defined as LncHIFCAR (long noncoding HIF-1α co-activating RNA). The invention also uncovers the up-regulation of LncHIFCAR/MIR31HG in oral carcinoma and the clinical relevance of LncHIFCAR as an independent adverse prognostic predictor for the cancer progression. Given its significance in the HIF-1 signaling pathway, LncHIFCAR/MIR31HG represents a novel and potential therapeutic target for the treatment of oral carcinoma.

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

As used herein, the term “diagnosis” refers to a process of identifying a disease by its signs, symptoms and results of various tests. The conclusion reached through that process is also called “a diagnosis.” In some examples, a diagnosis includes determining whether a tumor is benign or malignant. In other examples, a diagnosis includes determining whether a subject with cancer has a good or poor prognosis.

As used herein, the term “prognosis” refers to the prediction of the likelihood of cancer-attributable death or progression, including recurrence, metastatic spread, and drug resistance, of a neoplastic disease. Poor prognosis can refer to any negative clinical outcome, such as, but not limited to, a decrease in likelihood of survival (such as overall survival, relapse-free survival, or metastasis-free survival), a decrease in the time of survival (e.g., less than 5 years, or less than one year), presence of a malignant tumor, an increase in the severity of disease, a decrease in response to therapy, an increase in tumor recurrence, an increase in metastasis, or the like. In particular examples, a poor prognosis is a decreased chance of survival.

As used herein, the term “independent Prognostic Factor” denotes the independent nature of a given prognostic factor is established by multivariate statistical analysis delineating its independence from other prognostic factors. Independent prognostic factors can be particularly useful in clinical medicine since, by their independence, they can be applied to various clinical scenarios and they can be relied upon even in the absence of other clinical information. As a consequence, the identification of independent prognostic factors is a major focus of oncologic study.

As used herein, the term “prediction” refers to the likelihood that a patient will respond either favorably or unfavorably to a drug or set of drugs, and also the extent of those responses, or that a patient will survive, following surgical removal or the primary tumor and/or chemotherapy for a certain period of time without cancer recurrence. The predictive methods of the present invention can be used clinically to make treatment decisions by choosing the appropriate treatment modalities for any particular patient. The predictive methods of the present invention are valuable tools in predicting if a patient is likely to respond favorably to a treatment regimen, such as surgical intervention, chemotherapy with a given drug or drug combination, and/or radiation therapy, or whether long-term survival of the patient, following surgery and/or termination of chemotherapy or other treatment modalities is likely.

As used herein, the term “expression” refers to the process by which the coded information of a gene is converted into an operational, non-operational, or structural part of a cell, such as the synthesis of a protein.

As used herein, the terms “overexpress”, “overexpression”, “overexpressed”, “up-regulate”, or “up-regulated” interchangeably refer to a biomarker that is transcribed or translated at a detectably greater level, usually in a cancer cell, in comparison to a non-cancer cell or cancer cell that is not associated with the worst or poorest prognosis. The term includes overexpression due to transcription, post transcriptional processing, translation, post-translational processing, cellular localization, and/or RNA and protein stability, as compared to a non-cancer cell or cancer cell that is not associated with the worst or poorest prognosis.

The terms “subject” and “individual” are defined herein to include animals, such as mammals, including, but not limited to, primates, cows, sheep, goats, horses, dogs, cats, rabbits, guinea pigs, rats, mice or other bovine, ovine, equine, canine, feline, rodent, or murine species. In a preferred embodiment, the animal is a human.

As used herein, the term “recurrence-free survival” includes (1) any recurrence (local or regional (including invasive ipsilateral tumor and invasive locol regional tumor), or distant) and (2) death due to any cause (both BC and non-BC causes of death).

As used herein, the terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

As used herein, the terms “piRNA” and “Piwi-interacting RNA” are interchangeable and refer to a class of small RNAs involved in gene silencing. PiRNA molecules typically are between 26 and 31 nucleotides in length.

As used herein, the terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included.

As used herein, the term “biomarker” refers to a nucleic acid molecule which is present in a sample taken from interest subjects having human cancer as compared to a comparable sample taken from control subjects (e.g., a person with a negative diagnosis or undetectable cancer, normal or healthy subject).

As used herein, the term “risk” refers to the estimated chance of getting a disease during a certain time period.

As used herein, the term “treatment” is an intervention performed with the intention of preventing the development or altering the pathology or symptoms of a disorder.

As used herein, the term “level” refers to an absolute quantification of a molecule or an analyte in a sample, or to a relative quantification of a molecule or analyte in a sample, i.e., relative to another value such as relative to a reference or control taught herein, or to a range of values for the biomarker. These values or ranges can be obtained from a single subject or from a group of subjects.

In one aspect, the invention provides a method of diagnosing whether a subject has, or is at risk for a cancer, a metastatic cancer or a primary cancer, comprising:
a) isolating a LncHIFCAR transcript in a biological sample from the subject;
b) measuring a test level of the isolated LncHIFCAR transcript;
c) comparing the test level to a control level of the LncHIFCAR transcript; and
d) determining a subject as having the cancer, metastatic cancer or primary cancer when the test level is higher than the control level.

In one embodiment, the method further includes a step of administering a siRNA silencing LncHIFCAR to treat the cancer. In some embodiments, the siRNA comprises a sequence selected from the group consisting of the following:

(SEQ ID NO: 1)
GGGUUUCUGUAUUCAGUUATT;
(SEQ ID NO: 2)
UAACUGAAUACAGAAACCCTT;
(SEQ ID NO: 3)
CCAGCUGCUGAUGACGUAATT;
(SEQ ID NO: 4)
UUACGUCAUCAGCAGGUGGTT;

or its modified form.

The modified form of the siRNA includes, but is not limited to, 2′OMe nucleotides, 2′F nucleotides, 2′-deoxy nucleotides, 2′OMOE nucleotides, LNA nucleotides, and mixtures thereof. In preferred embodiments, the modified nucleotide comprises a 2′OMe nucleotide (e.g., 2′OMe purine and/or pyrimidine nucleotide) such as, for example, a 2′OMe-guanosine nucleotide, 2′OMe-uridine nucleotide, 2′OMe-adenosine nucleotide, 2′OMe-cytosine nucleotide, and mixtures thereof. In certain instances, the modified nucleotide is not a 2′OMe-cytosine nucleotide.

The siRNA can be prepared in many ways such as chemical synthesis, in vitro transcription, enzyme cleavage of long-chain dsRNA, vector expression of siRNA, PCR synthesis of siRNA expression elements. The presence of these methods provides a selection space for researchers and can be used to obtain better gene silencing efficiency.

The diagnosis described herein includes a diagnosis in various stages of a cancer. Examples of the stages include, but are not limited, early stage, invasion stage and metastatic stage of a cancer.

In another aspect, the invention provides a method of determining a prognosis, recurrence-free survival or overall survival of a subject having, or suspected of a cancer, a metastatic cancer or a primary cancer, comprising: a) isolating a LncHIFCAR transcript in a biological sample from the subject; b) measuring a test level of the isolated LncHIFCAR transcript; c) comparing the test level to a control level of the LncHIFCAR transcript; and d) determining a subject as having a poor prognosis, poor recurrence-free survival or poor overall survival when the test level is higher than the control level.

In one embodiment, the determination of a prognosis can be used as an independent prognostic factor.

In one embodiment, the method further includes a step of administering a siRNA silencing LncHIFCAR to treat the cancer. In some embodiments, the siRNA comprises a sequence of SEQ ID NO:1, 2, 3 or 4.

The level of LncHIFCAR transcript described herein can be measured in cells of a biological sample obtained from the subject. In some embodiments, the biological sample is a sample of tissue or fluid isolated from a subject, including but not limited to, for example, urine, blood, plasma, serum, fecal matter, bone marrow, bile, spinal fluid, lymph fluid, external secretions of the skin, respiratory, intestinal, and genitourinary tracts, tears, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components. A corresponding control tissue or blood sample, or a control reference sample, can be obtained from unaffected tissues of the subject, from a normal human individual or population of normal individuals, or from cultured cells corresponding to the majority of cells in the subject's sample. The control tissue or blood sample is then processed along with the sample from the subject, so that the levels of LncHIFCAR from the subject's sample can be compared to the corresponding LncHIFCAR from the control sample. Alternatively, a control sample can be obtained and processed separately (e.g., at a different time) from the test sample and the level of LncHIFCAR produced from the sample can be compared to the corresponding LncHIFCAR level from the control sample.

The LncHIFCAR can be detected and quantitated by a variety of methods including, but not limited to, microarray analysis, polymerase chain reaction (PCR), reverse transcriptase polymerase chain reaction (RT-PCR), real time reverse transcriptase polymerase chain reaction (RT-PCR), quantitative real time polymerase chain reaction (qPCR), Northern blot, serial analysis of gene expression (SAGE), immunoassay, and mass spectrometry, any sequencing-based methods known in the art.

An increase in the level of LncHIFCAR in the sample obtained from the subject, relative to the level of a corresponding LncHIFCAR in a control sample, is indicative of the methods of the invention. The relative LncHIFCAR in the control samples can be determined with respect to one or more RNA expression standards. The standards can comprise, for example, the LncHIFCAR level in a standard cell line, the LncHIFCAR level in unaffected tissues of the subject.

In some embodiments, the test level and the control level may be expressed as a mean comparative quantification (Cq) test value and a mean comparative quantification (Cq) control value (delta Cq method). In such a case, the mean Cq test value and a mean Cq control value are normalized by an internal control.

In one embodiment, for determining the prognosis, recurrence-free survival or overall survival of a subject is, or is suspected of a cancer or diagnosing whether a subject has, or is at risk for developing a cancer, a metastatic cancer or a primary cancer, the level of the LncHIFCAR in the sample is greater than the reference level in the control sample.

In certain embodiments, the gene expressing LncHIFCAR used in the above methods is MIR31HG whose sequence is disclosed in NCBI Reference Sequence: NR_027054.1, NR_027054.2, NR_152877.1, NR_152878.1 or NR_152879.1.

In another further aspect, the invention provides a kit for predicting a risk for developing a cancer, a metastatic cancer or a primary cancer or a prognosis, recurrence-free survival or overall survival of a subject, comprising reagents for determining a level of the LncHIFCAR in the sample. The kit is assemblage of reagents for measuring RNA. It is typically in a package which contains all elements, optionally including instructions. The package may be divided so that components are not mixed until desired. The kit may contain reagents for determining RNA level.

In another aspect, the invention provides a method of treating a cancer, a metastatic cancer or a primary cancer in a subject comprising administering to the subject an effective amount of a therapeutic agent that blocks an expression or overexpression of MIR31HG gene or a physiological action of a LncHIFCAR transcript. In some embodiments, the therapeutic agent is selected from an antisense oligonucleotide, an antisense RNA, a small molecular inhibitor, an antisense cDNA, RNA, siRNA, esiRNA, shRNA, miRNA, decoy, RNA aptamer, RNA/DNA demethylating agent and RNA/DNA-binding protein/peptide or a compound to inhibit one or more physiological actions affected by LncHIFCAR. In one embodiment, the therapeutic agent is a siRNA silencing LncHIFCAR. In some embodiments, the siRNA comprises a sequence of SEQ ID NO: 1, 2, 3 or 4, or its modified form.

In some embodiments, the cancers described herein, including a tumor, metastasis, or other disease or disorder characterized by uncontrolled cell growth. In some embodiments, the cancers include, but are not limited to, oral cancer (such as an oral squamous cell carcinoma (OSCC)) or a hypoxia-mediated oral cancer), brain cancer (such as glioblastoma), kidney cancer (such as kidney renal clear cell carcinoma) or a hypoxia-mediated brain cancer, colorectal cancer or a hypoxia-mediated colorectal cancer, or uterine cancer (such as uterine corpus endometrial carcinoma) or a hypoxia-mediated uterine cancer. In other embodiments, the cancers described herein, carcinomas, myelomas, melanomas or gliomas may be treated.

All publications and patent documents cited in this application are incorporated by reference in their entirety for all purposes to the same extent as if each individual publication or patent document were so denoted. By their citation of various references in this document, Applicants do not admit any particular reference is “prior art” to their invention.

### Example

### Methods

Cell Culture

Cell lines HeLa and HEK293T (293T) were obtained from ATCC and cultured in DMEM supplemented with 10% (v/v) FBS (Gibco/Invitrogen). SAS cell line was obtained from JCRB cell bank and maintained in 1:1 mixture of DMEM and Ham's F12 medium (Gibco/Invitrogen) with 10% FBS. The oral cancer cell lines OC-2, OEC-M1 and HSC-3 were obtained and maintained as described previously. Cell lines were not authenticated, but regularly tested for mycoplasma contamination using Venor GeM Detection Kit (Minerva Biolabs). None of the cell line stock used in this study is found in the database of commonly misidentified cell lines listed by ICLAC. Transfection was performed using Lipofectamine 2000 (Invitrogen) or Fugene 6 (Roche Applied Science) as recommended by the manufacturer. To generate stable LncHIFCAR-knockdown SAS cell lines, SAS cells transduced with LncHIFCAR shRNA (sh-HIFCAR) or empty vector (sh-Ctrl) lentiviral particles were selected by Zeocin for 3 weeks. To generate luciferase-expressing and LncHIFCAR-knockdown SAS cell lines for xenograft study, the SAS stable clones (sh-Ctrl #1 and sh-HIFCAR #6) described above were transduced with a lentiviral vector containing firefly luciferase cDNA, which was constructed using the ViralPower Lentiviral Gateway Expression System from Invitrogen and selected by puromycin for 3 weeks. For hypoxia treatment, physical hypoxic conditions (1% oxygen) were generated by Forma™ Series II 3130 incubators (Thermo Scientific). Alternatively, to generate chemical-induced pseudo-hypoxic state, cells were treated with hypoxia-mimetic chemical cobalt chloride (Sigma-Aldrich) as indicated.

Real Time qRT-PCR

Total cellular RNA was isolated using Trizol (Invitrogen) reagent, and cDNA was generated using SuperScript II first-strand synthesis system (Invitrogen). Real-time quantitative PCR analysis was performed on the Bio-Rad iQ5 Real-Time PCR detection system (Bio-Rad) with Maxima SYBR Green qPCR Master Mix (Fermentas) or on the Rotor-Gene Q instrument (Qiagen) with QuantiFast SYBR Green PCR Kit (Qiagen). All expression levels, unless otherwise specified, were normalized against the GAPDH mRNA level. The primer sequences are listed in Supplementary Data 2.

Supplementary Data 2.
Primers used for PCR in the present study

| Primer name | Sequence (5′ to 3′) |
|---|---|
| LncHIFCAR | F: GTTTCTGGTCCTCATACCGTGTGGTT |
| | R: CTTGGAATGAATCCTCTGTCCTCC |
| CA9 | F: TGAGGAAGGCTCAGAGACTCA |
| | R: GAGGCCAAAAACCAGGGCTA |
| actin | F: CATGTACGTTGCTATCCAGGC |
| | R: CTCCTTAATGTCACGCACGAT |
| 18S rRNA | F: CGGCGACGACCCATTCGAAC |
| | R: GAATCGAACCCTGATTCCCCGTC |
| 28S rRNA | F: CCCAGAAAAGGTGTTGGTTG |
| | R: ATGGAACCCTTCTCCACTTC |
| RPLP0 | F: TGGTCATCCAGCAGGTGTTCGA |
| | R: ACAGACACTGGCAACATTGCGG |
| GAPDH | F: TCCACTGGCGTCTTCACC |
| | R: GGCAGAGATGATGACCCTTTT |
| RMRP | F: TGCTGAAGGCCTGTATCCT |
| | R: TGAGAATGAGCCCCGTGT |
| MALAT1 | F: GGATCCTAGACCAGCATGCC |
| | R: AAAGGTTACCATAAGTAAGTTCCAGAAAA |
| PCAT1 | F: ACCAGTGGAGAAGAGGCAGA |
| | R: GCTTCAAATGCGAAAAGACC |
| HIF-1α | F: TGCTTGCCAAAAGAGGTGGA |
| | R: TTCTGTGTCGTTGCTGCCAA |
| Glut1 | F: CGGGCCAAGAGTGTGCTAAA |
| | R: TGACGATACCGGAGCCAATG |
| SOD2 | F: GCTCCGGTTTTGGGGTATCTG |
| | R: GCGTTGATGTGAGGTTCCAG |
| LOXL2 | F: GGAAAGCGTACAAGCCAGAG |
| | R: GCACTGGATCTCGTTGAGGT |
| PDK1 | F: ACCAGGACAGCCAATACAAG |
| | R: CCTCGGTCACTCATCTTCAC |
| LDHA | F: ATCTTGACCTACGTGGCTTGGA |
| | R: CCATACAGGCACACTGGAATCTC |
| L1CAM | F: GCCACCTGTCATCACGGAAC |
| | R: GTCCAGCGGAACTGCACTTC |
| BNIP3 | F: AACTCAGATTGGATATGGGATTGG |
| | R: AGAGCAGCAGAGATGGAAGG |
| CITED | F: TTGGACCGCATCAAGGAG |
| | R: ATTCACGCCGAAGAAGTTG |
| RPL13A | F: CTCAAGGTCGTGCGTCTG |
| | R: TGGCTTTCTCTTTCCTCTTCTC |
| VEGF | F: CGCAAGAAATCCCGGTATAA |
| | R: TCTCCGCTCTGAGCAAGG |
| LncHIFCAR | F: GGTCTGCTTGTATTCAATGACTGGTC |
| (Region A) | R: CTGCAGTGTAGTCACAAAATGGCCT |
| LncHIFCAR | F: TGCCAGTAGAGGGAAGAGGA |
| (Region B) | R: TCCTGTCAGATCAGCAGTGG |
| ChIP-actin | F: TTCTACGTTTCCATCCAAGCCGT |
| | R: TTTCTTGTTCGAAGTCCAAGTCCAAGG |
| ChIP-LDHA | F: TTGGAGGGCAGCACCTTACTTAGA |
| | R: GCCTTAAGTGGAACAGCTATGCTGAC |
| ChIP-VEGF | F: AGACTCCACAGTGCATACGTG |
| | R: AGTGTGTCCCTCTGACAATG |
| ChIP-Glut1 | F: GGGCTGTGTTACTCACTCTTACTCC |
| | R: CTCTTCCTGGGTTGTGTTCAAGCTG |
| ChIP-PDK1 | F: CGCGTTTGGATTCCGTG |
| | R: CCAGTTATAATCTGCCTTCCCTATTATC |
| ChIP-RPL13A | F: GAGGCGAGGGTGATAGAG |
| (non-HRE) | R: ACACACAAGGGTCCAATTC |

F, Forward; R, Reverse

Real time quantitative PCR for miR-31 was performed using miScript PCR Starter Kit and hsa-miR-31 miScript Primer Assay according to manufacturer's instructions (Qiagen).

Molecular Cloning and siRNA Transfection

To generate sense and anti-sense biotin-labelled LncHIFCAR transcripts, full-length LncHIFCAR cDNA was amplified from HeLa nuclear RNA and cloned into pCR2.1-TOPO (Invitrogen) by TA cloning. For in vitro transcription of biotin-labelled LncHIFCAR RNA deletion variants, the corresponding LncHIFCAR fragments were amplified and cloned into pGemT-Easy (Promega) by TA cloning. For ectopic overexpression of full-length RMRP, LncHIFCAR and LncHIFCAR (501-1500) in human cell lines, the PCR-generated DNA fragments containing the indicated regions were inserted into pCR2.1-TOPO by TA cloning, followed by sub-cloning into the EcoRI site of pSL-MS2 vector. To knockdown LncHIFCAR expression, DNA encoding shRNA specifically targeting LncHIFCAR at sequence GCTGCTGATGACGTAAAGT was cloned into pLenti4 vector. For siRNA-mediated knockdown of LncHIFCAR, two different siRNA oligonucleotides were synthesized and purified by Genepharma (Suzhou, Jiangsu, China). siRNAs were transfected at a final concentration of 20 nM using Lipofectamine RNAiMAX Reagent (Invitrogen) following the manufacturer's protocol. The sequences of siRNAs are listed in Supplementary Table 2.

Supplementary Table 2.
List of siRNA sequences

| Probe name | Sequence (5′ to 3′) | Reference |
|---|---|---|
| Negative Control | Sense: UUCUCCGAACGUGUCACGUTT | |
| (NC) | Antisense: ACGUGACACGUUCGGAGAATT | |
| si-HIFCAR-1 | Sense: GGGUUUCUGUAUUCAGUUATT | Oncogene 35, |
| | Antisense: UAACUGAAUACAGAAACCCTT | 3647-3657 (2016) |
| si-HIFCAR-2 | Sense: CCAGCUGCUGAUGACGUAATT | |
| | Antisense: UUACGUCAUCAGCAGCUGGTT | |

The HIF-1α-expressing plasmid HA-HIF-1α-pcDNA3, as well as the reporter plasmid HRE-luciferase (HRE-FLuc) containing hypoxia-response elements (HREs) fused with a firefly luciferase, were purchased from Addgene. The pRL-SV40 luciferase constitutive reporter plasmid were purchased from Promega. To express and purify GST-HIF-1α, the PCR-amplified HIF-1α fragments from HA-HIF-1α-pcDNA3 was inserted into pGemT-Easy by TA cloning, followed by sub-cloning into the NotI site of pGEX-6p-1 plasmid (GE Healthcare). To purify GST-HIF-1α deletion variants, the corresponding amplified HIF-1α fragments were cloned into the BamHI/NotI sites of pGEX-6p-1. The integrity of each construct was verified by DNA sequencing and the sequences of specific primers designed for cloning. The pLK0.1-puro plasmid-based shRNAs, including TRCN0000003808 (HIF1A-sh1) and TRCN0000003809 (HIF1A-sh2), were obtained from the National RNAi Core Facility, Institute of Molecular Biology/Genomic Research Center, Academia Sinica, Taiwan.

cBioPortal and Oncomine Database Analysis

LncHIFCAR expression was analyzed using the Oncomine (www.oncomine.org) and cBioPortal (www.cbioportal.org) platforms. For the tumor versus normal analysis of LncHIFCAR (LOC554202) on Oncomine, the following datasets were used: Peng Head-Neck dataset (GEO: GSE25099)²⁵, Vasko Thyroid dataset (GSE6004), He Thyroid dataset (GSE3467), Zhao Breast dataset (GSE3971) and TCGA colorectal dataset (TCGA Research Network; http://cancergenome.nih.gov/). The p value smaller than 0.05 was considered statistically significant. For LncHIFCAR/MIR31HG expression analysis and co-expression network discovery, the TCGA head and neck squamous cell carcinoma cohort (TCGA, Provisional) was analyzed on cBioPortal using the default options according to the instructions on the website. Pearson and Spearman correlations of the expression levels of 20532 genes in 522 HNSCC cases were accessed and computed by RNA-Seq V2 RSEM dataset. By default, only gene pairs with values >0.3 or <−0.3 in both measures are considered statistically significant and shown. Total 248 significantly co-expressed genes were listed, including LCAM and LDHA.

Cell Migration and Invasion Assay

3×10⁴ cells were suspended in 100 μl of DMEM without FBS and seeded into the top chamber of 24-well plate-sized transwell inserts (BD Falcon, 353097) with a membrane of 8 μm pore size. The medium containing 10% FBS was placed into the lower chamber as a chemoattractant. After incubation for 24 hours, the cells that did not migrate through the pores were manually removed with a cotton swab. Cells presented at the bottom of the membrane were fixed and stained with crystal violet and then counted and imaged under microscope. Cell numbers were calculated in eight random fields for each chamber, and the average value was calculated. Each experiment was conducted in triplicate. Matrigel invasion assays were performed using Matrigel-coated transwell inserts with the procedure as described above.

Sphere Formation Assay

Single-cell suspensions of SAS cells were plated (1000 cells per well) into 6-well Ultra Low Attachment plates (Corning) in serum-free DMEM/F12 culture media (Gibco/Invitrogen) supplemented with 2% B27 (Invitrogen), 20 ng mL⁻¹ bFGF (Invitrogen), and EGF (20 ng mL⁻¹, Millipore). The cells were grown in a humidified atmosphere of 95% air and 5% CO₂ for 15 days. Upon harvest, the spheres were counted (diameter >100 μm) with inverted phase contrast microscopy, followed by collection for RNA extraction. For pimonidazole staining, tumor spheres grown in normoxic suspension culture were allowed to attach to 0.1% gelatin-coated cover slips for 12 hours. Pimonidazole (Hypoxyprobe™-1 Kit, Hypoxyprobe, Burlington, USA) was applied to the spheres for 1 hour under normoxia. Intracellular pimonidazole complexes indicative of hypoxic conditions were detected by immunofluorescence microscopy using an anti-pimonidazole monoclonal antibody (1:200, Hypoxyprobe™-1 Kit). Cell nuclei were counterstained by Hoechst staining whereas necrotic cells were labeled with propidium iodide (PI) fluorescence staining. For quantification of necrotic spheres, suspended to tumor spheres were collected by centrifugation for the subsequent PI fluorescence staining.

Luciferase Reporter Assay

The HIF-1α-responsive luciferase construct (pHRE-FLuc) containing hypoxia-response elements (HREs) fused with a firefly luciferase was purchased from Addgen. For HRE luciferase assays, cells were seeded to 24-well plates at a density of 1×10⁵ per well. After overnight incubation, cells were transiently co-transfected with the pHRE-FLuc reporter plasmid, empty vector, LncHIFCAR-expressing constructs or shRNA vector-targeting LncHIFCAR, as well as an internal control construct pRL-SV40 Renilla luciferase plasmid (Promega). 24 hours post transfection, the media was replaced and the cells were exposed to 20% or 1% 02 for 24 hours. At 48 hours post-transfection, cells were lysed with passive lysis buffer and assayed for firefly and Renilla luciferase activities using Varioskan Flash microplate luminometer (Thermo) with the Dual-Luciferase Assay System (Promega). All the luciferase activity were normalized against the Renilla values and expressed as the relative fold of control group.

Glucose Uptake and Lactate Production Assay

The intracellular glucose and extracellular lactate were measured with the fluorescence-based glucose assay and lactate assay kits (BioVision) according to the manufacturer's instructions, respectively. Vector control and LncHIFCAR-knockdown SAS clones were cultured for 24 hours following subsequent treatment of normoxia or hypoxia for 16 hours. Intracellular glucose levels and lactate levels in the culture media were measured and presented as folds relative to the level of control cells in normoxia. All measurement were normalized by cell number.

Nuclear and Cytoplasmic Fractionation

Subcellular fractionation of protein extracts from HeLa cells was performed using NE-PER Nuclear and Cytoplasmic Extraction Reagents (Thermo Scientific) following the manufacturer's protocol. Nuclear/cytoplasmic fractionation of RNA was conducted with Nuclei EZ Lysis Buffer (Sigma), according to the manufacturer's protocol.

Biotinylated RNA Pull-Down

The biotinylated RNA pull-down assay was performed as described previously. Briefly, biotin-labeled RNAs was in vitro transcribed with AmpliScribe T7-Flash Biotin-RNA Transcription Kit (Epicentre), treated with RNase-free DNase I and purified with an RNeasy Mini Kit (Qiagen). The lambda transcript was generated with the control plasmid provided by the Transcription Kit. To form the proper secondary structure, biotinylated RNA supplied with RNA structure buffer (10 mM Tris pH 7, 0.1 M KCl and 10 mM MgCl₂) was heated to 90° C. for 2 minutes, incubated on ice for 2 minutes, and then shifted to room temperature (RT) for 20 minutes. The RNA was then mixed with hypoxic HeLa nuclear extract or purified proteins and incubated at RT for one hour, followed by incubating with Streptavidin Mag Sepharose (GE Healthcare) at RT for one hour. After subsequent washes, the pull-down complexes were analyzed by standard western blot technique.

Antibodies and Western Blot Analysis

Cells were harvested, rinsed with PBS and lysed in lysis buffer (1% NP-40, 150 mM NaCl, 50 mM Tris-HCl pH 7.4, 1 mM EDTA, 1 mM MgCl₂, 0.5% NP-40, 1 mM Na₃VO₄, 1 mM NaF, protease inhibitors cocktail). Cell lysates were separated on SDS-polyacrylamide gel, transferred to a PVDF membrane (Bio-Rad Laboratories) and immunoblotted using the following primary antibodies. Rabbit anti-HIF-1α (1:1000, GTX127309), anti-GST (1:5000, GTX110736), anti-lamin B2 (1:5000, GTX109894) and anti-tubulin (1:5000, GTX112141) antibodies, as well as mouse monoclonal anti-HIF-1α antibody (1:1000, GTX628480), were purchased from GeneTex. Mouse monoclonal antibodies recognizing β-actin (1:5000, A2228) were purchased from Sigma. Mouse monoclonal HIF-1β antibody [2B10] (1:2000, ab2771) were purchased from Abcam. Uncropped scans of the blots and gels are shown in Supplementary FIG. 19 in the Supplementary Information section.

Purification of GST-HIF-1α

E. coli host BL21(DE3) harboring the expression vector pGEX-6p-1-HIF-1α was cultured in Luria-Bertani medium with ampicillin (50 μg ml⁻¹) and induced by 0.3 mM IPTG (isopropyl-β-D-thiogalactopyranoside) at 30° C. for 16 hours. Affinity purification of the recombinant protein was carried out with Pierce Glutathione Superflow Agarose (Pierce) following the manufacturer's instructions.

RNA Immunoprecipitation

RNA immunoprecipitation (RIP) was performed with the following modifications. Briefly, 2×10⁷ of SAS cells treated with normoxia or hypoxia for 16 hours were crosslinked with 0.3% formaldehyde in medium for 10 min at 37° C., followed by neutralization with 125 mM glycine incubated at room temperature for 5 min. After two times wash with cold PBS, the cell pellets were lysed in RIPA buffer (50 mM Tris, pH 7.4, 150 mM NaCl, 1 mM EDTA, 0.1% SDS, 1% Nonidet P-40, and 0.5% sodium deoxycholate, 0.5 mM DTT, RNase inhibitor and protease inhibitor cocktail), followed by sonication on ice and subsequent DNase treatment for 30 min. immunoprecipitation were performed by incubating protein A/G precleared nuclear lysates with a-HIF-1α antibody (1:100, NB100-105, Novus Biologicals) or equivalent mouse IgG (GTX35009, GeneTex) at 4° C. overnight. The RNA/antibody complex was then precipitated by incubation with protein A/G agarose beads. After subsequent wash following the standard protocol, the RNA samples were extracted with Trizol reagent (Invitrogen) and detected by qRT-PCR.

Immunoprecipitation

Vector control or LncHIFCAR knockdown SAS cells under hypoxic conditions for 24 hours were collected by centrifugation. The cell pellet was then resuspended in lysis buffer (50 mM Tris-HCl pH 8.0, 180 mM NaCl, 1% NP-40, protease and phosphatase inhibitor cocktail) and passed through a 21-gauge syringe several times. Immunoprecipitations were performed by incubating 1 mg protein A/G precleared cell lysates with 5 μg a-HIF-1α antibody (NB100-105, Novus Biologicals) or equivalent mouse IgG (GTX35009, GeneTex) at 4° C. for 3 hours. After subsequent wash, the immunoprecipated protein complex on the beads were analyzed by western blotting.

In Vitro RNA-Binding Assay

In vitro synthesized LncHIFCAR RNA supplied with RNA structure buffer (10 mM Tris pH 7, 0.1 M KCl and 10 mM MgCl₂) was heated to 90° C. for 2 minutes, incubated on ice for 2 minutes, and then shifted to room temperature for 20 minutes to form the proper secondary structure. GST fusion proteins on glutathione-Sepharose beads (˜10 μl) were incubated with 2 μg in vitro synthesized LncHIFCAR RNA in 50 μl of RNA-binding buffer (50 mM Tris-HCl, pH 7.4, 100 mM KCl, 2 mM MgCl₂, 0.1% NP-40. 1 mM DTT and ribonuclease inhibitor) for 30 min at 4° C. The beads were washed with RNA-binding buffer 3 times to remove unbound RNAs. The RNA samples retained on the beads were extracted with Trizol reagent (Invitrogen) and detected by qRT-PCR. The relative retention as to the input RNA level was calculated.

Chromatin Immunoprecipitation

Chromatin immunoprecipitation (ChIP) assays were performed mainly following the ChIP protocol from the University of California Davis Genome Center (http://farnham.genomecenter.ucdavis.edu). Briefly, vector control or LncHIFCAR knockdown SAS cell lines (2×10⁷ cells per assay) treated with normoxia or hypoxia were cross-linked with 1% formaldehyde at 37° C. for 15 min and subsequently quenched in 125 mM glycine for 5 min. The cross-linked chromatin was sonicated to generate DNA fragments averaging 200-500 bp in length. Chromatin fragments were immunoprecipitated with antibodies against HIF-1α (1:100, NB100-105, Novus Biologicals), p300 (1:100, 05-257, Millipore), or equivalent mouse IgG (GTX35009, GeneTex). The precipitated DNA were purified using the QIAquick PCR Purification Kit (Qiagen) and analyzed by quantitative real-time PCR using the primers listed in Supplementary Data 2.

Chromatin Isolation by RNA Purification

Chromatin isolation by RNA purification (ChIRP) was performed using SAS cells adapting the protocols with minor modifications. Briefly, 20-mer antisense DNA probes targeting LncHIFCAR RNA, as well as the negative control lacZ RNA, were designed using the online probe designer at singlemoleculefish.com (http://www.singlemoleculefish.com/designer.html) as listed in Supplementary Table 4.

Supplementary Table 4.
Probe sequences used in the ChIRP

| Probe name | Sequence (5′ to 3′) |
|---|---|
| LncHIFCAR | |
| ChIRP LncHIFCAR-1 | TCCGAGTAGGAGGACAGAAG |
| ChIRP LncHIFCAR-2 | TTGTGTCCACAACACATTCT |
| ChIRP LncHIFCAR-3 | TATTTCCAGGAATCCATCTC |
| ChIRP LncHIFCAR-4 | ACACTTTACGTCATCAGCAG |
| ChIRP LncHIFCAR-5 | TCTTTCCTCTATGATGTGTT |
| ChIRP LncHIFCAR-6 | CCTTCTTGTGTCTAAAGGAC |
| ChIRP LncHIFCAR-7 | TAGGATATAACCTGCCTCAG |
| ChIRP LncHIFCAR-8 | GCCAAAAGCATCCTGATTTC |
| ChIRP LncHIFCAR-9 | CTCCATTAAAGCCATGCATA |
| ChIRP LncHIFCAR-10 | CCTCCTTTTAGGTCATATAG |
| ChIRP LncHIFCAR-11 | GTTTCTCATCTGATTGATCA |
| ChIRP LncHIFCAR-12 | GATCCTGATTTCCTATGCAA |
| ChIRP LncHIFCAR-13 | CCATCAACGTCTTCTGTGAA |
| ChIRP LncHIFCAR-14 | CCAGGGAAGCATAACCACAT |
| ChIRP LncHIFCAR-15 | TTTCTTTTAGGGGTATTGGC |
| ChIRP LncHIFCAR-16 | GTGAATCATCACTGCTGAGG |
| ChIRP LncHIFCAR-17 | AAGAAGCAAGAACCTCCCTG |
| lacZ | |
| ChIRP lacZ-1 | TAGCCAGCTTTCATCAACAT |
| ChIRP lacZ-2 | AGCAGCAGACCATTTTCAAT |
| ChIRP lacZ-3 | GTGTGGGCCATAATTCAATT |
| ChIRP lacZ-4 | CGGCAGCCGTTATTATTATT |
| ChIRP lacZ-5 | GAAACTGTTACCCGTAGGTA |
| ChIRP lacZ-6 | CACGGCGTTAAAGTTGTTCT |
| ChIRP lacZ-7 | GGATCGACAGATTTGATCCA |
| ChIRP lacZ-8 | GTAGTTCAGGCAGTTCAATC |
| ChIRP lacZ-9 | CAACGGTAATCGCCATTTGA |
| ChIRP lacZ-10 | TGCAAGGCGATTAAGTTGGG |

SAS cells treated with normoxia or hypoxia for 4 hours were crosslinked with 1% glutaldehyde for 10 min at room temperature with gentle shaking. Crosslinking was stopped with 125 mM glycine for 5 min. The cross-linked chromatin was incubated with the biotinylated DNA probes, subjected to streptavidin magnetic beads capturing and subsequent wash/elution steps essentially performed as described⁷⁰. The eluted chromatin and RNA fragments were analyzed by qPCR using the primers listed in Supplementary Data 2.

### Xenograft Experiments

Oral cancer SAS cell lines (sh-Ctrl#1 and sh-HIFCAR#6) used for metastasis model were all labeled with firefly luciferase as described above. In brief, 5×10⁵ SAS cells (sh-Ctrl#1 and sh-HIFCAR#6, respectively) suspended in 0.5 ml of Matrigel (BD Bioscience) were injected into the tail veins of 6- to 8-week-old female athymic nude mice (nu/nu). Subsequently, the mice were monitored for metastases using the IVIS Lumina LT series III system (PerkinElmer) after intraperitoneal injection of luciferin once a week for 6 weeks after tail vein xenografting. Then, the lungs of the nude mice were excised post mortem for histology examination and hematoxylin-eosin staining. The experiments were not randomized. The investigators were not blinded to allocation during experiments and outcome assessment. The animal studies were approved by National Health Research Institutes Institutional Animal Care and Use Committee (approval number: NHRI-IACUC-102078), and carried out under the institutional guidelines with animal welfare standards.

Patients and Clinical Samples

The research design, study protocols and information security were approved by the Institutional Review Board of Chi-Mei Medical Center (approval number: 10312-L07). Thereby, snap-frozen primary OSCC tissues and paired noncancerous mucosa tissues stored in liquid nitrogen were withdrawn from Chi-Mei Medical Center Tissue BioBank. Written informed consents were obtained from all participants.

Statistical Analysis

All data were expressed as mean±SD of three or more independent experiments. Sample sizes were selected based on experience from our previous publications. Samples were excluded only in the case where a technical error occurred during sample preparation or analysis. Statistical analyses were performed with SPSS software version 19.0 (Armonk). Differences between individual groups were analyzed by 2-tailed Student's t test. The survival curves were calculated using the Kaplan-Meier method, and the differences were assessed by a log-rank test. Statistical analyses of clinicopathological data were performed using Fisher's exact test. Univariate and multivariate Cox proportional hazards regression models were performed to identify the independent factors with a significant impact on patient survival. The hazard ratios (HRs) and 95% confidence intervals of the prognostic factors were calculated. The results were considered significant if P <0.05.

Data Availability

The TCGA and Oncomine data referenced during the study are available in a public repository from the cBioPortal for Cancer Genomics, TCGA (http://www.cbioportal.org/) and the Oncomine (www.oncomine.org) websites. For the tumor versus normal analysis of LncHIFCAR on Oncomine (search term: LOC554202), the following datasets were used: Peng Head-Neck dataset (Gene Expression Omnibus: GSE25099), Vasko Thyroid dataset (GSE6004), He Thyroid dataset (GSE3467), Zhao Breast dataset (GSE3971) and TCGA colorectal dataset (TCGA Research Network; http://cancergenome.nih.gov/). For LncHIFCAR expression analysis and co-expression network discovery, the TCGA head and neck squamous cell carcinoma cohort (TCGA, Provisional) was analyzed on cBioPortal platform (search term: MIR31HG). The authors declare that the data supporting the findings of this study are included within the article and its Supplementary Information files, or are available from the authors upon reasonable request.

### Example 1 the Expression of lncRNA LncHIFCAR is Induced by Hypoxia

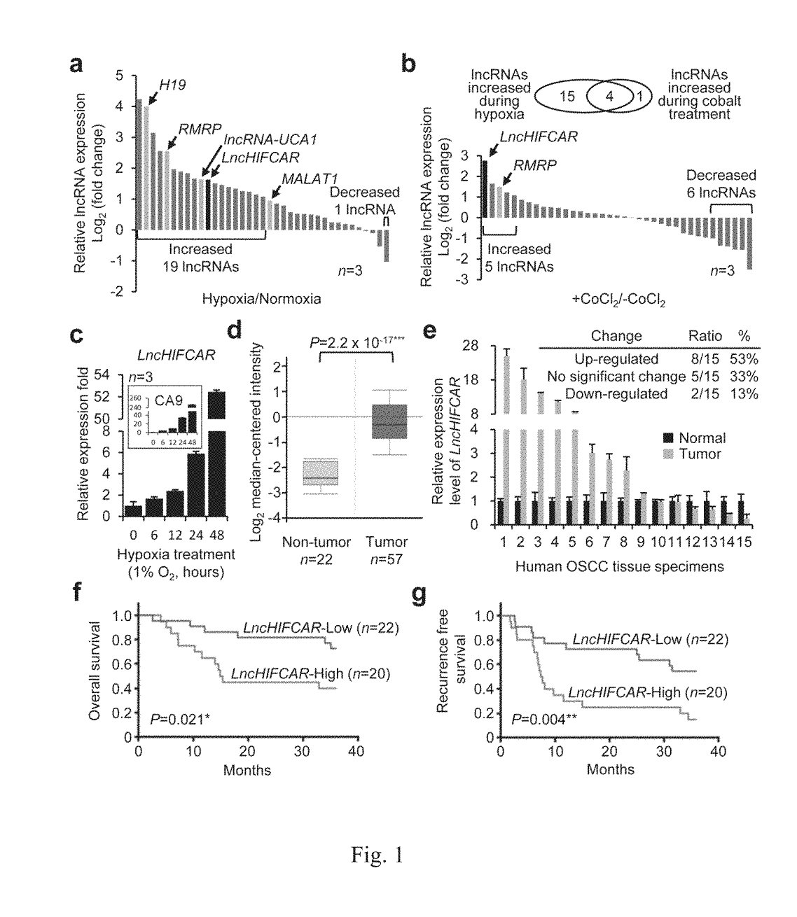

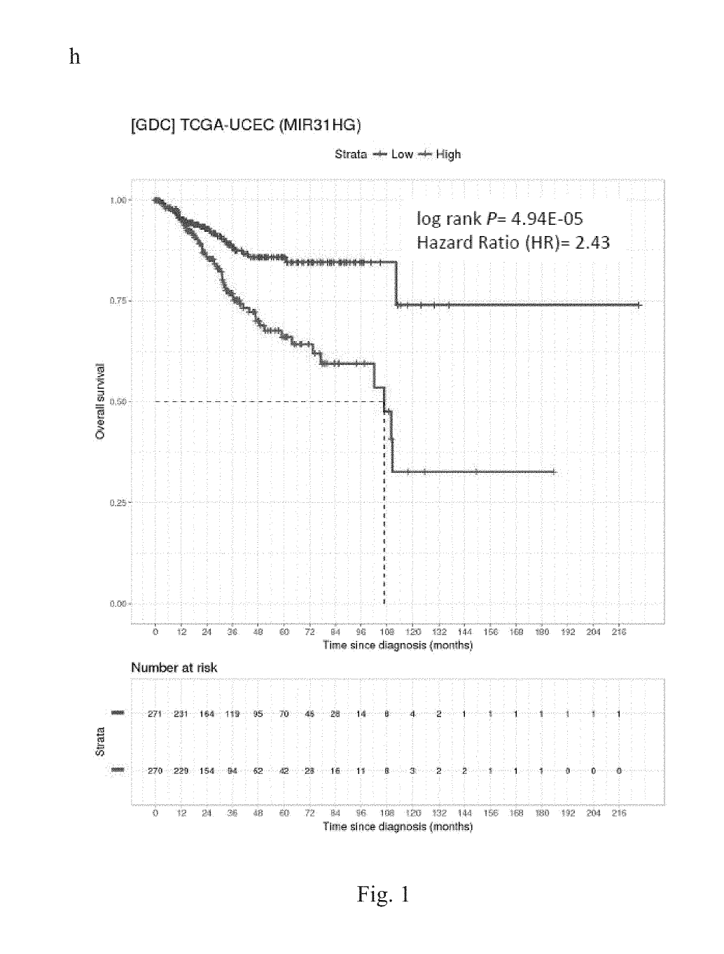

To identify lncRNAs involved in HIF-1 signaling pathway and hypoxia-associated cancer progression, we initially selected 37 cancer-associated lncRNAs according to previous reports and examined their expression profiles in HeLa cells before and after hypoxia treatment using quantitative real-time PCR (qRT-PCR). Validation of a panel of known hypoxia-inducible protein-coding genes by qRT-PCR confirmed the robustness of our screenings. Compared with non-hypoxic controls, several lncRNAs with a >2-fold alteration in expression were identified under hypoxic conditions. (FIG. 1a). Notably, several hypoxia-responsive lncRNAs reported by recent studies, such as H19 and lncRNA-UCA1, were also identified in this test. In addition, taking advantage of the hypoxia-mimetic agent cobalt chloride, known to stabilize HIF1α by inactivating prolyl hydroxylases and induce a cellular pseudo-hypoxic state, we examined the expression of the lncRNAs, and identified 5 of them up-regulated by >2-fold after treatment of cobalt chloride in HeLa cells (FIG. 1b). These lncRNAs include four of those induced in physical hypoxia. LncHIFCAR/MIR31HG was the most substantially up-regulated lncRNA in response to cobalt chloride treatment, and displayed a time- and dose-dependent induction. Similar to the chemically induced pseudo-hypoxic conditions, a time-dependent increase of LncHIFCAR was also observed in physical hypoxic HeLa cells (FIG. 1c), confirming LncHIFCAR as a specific, hypoxia-inducible lncRNA that is possibly involved in HIF-1α signaling pathway.

### Example 2 Upregulation of LncHIFCAR as a Prognostic Biomarker for OSCC

To evaluate the clinical significance of LncHIFCAR in cancer progression, we first queried the Oncomine database (www.oncomine.com) to systematically assess the relative LncHIFCAR expression in different cancer types (normal versus cancer). Several types of cancer were found to exhibit a significant up-regulation of LncHIFCAR, including oral squamous cell carcinoma (OSCC, P=2.2×10⁻¹⁷, Student's t test; FIG. 1d), colon adenocarcinoma, rectal adenocarcinoma, thyroid cancer and breast cancer. Consistent with the published OSCC dataset, in a panel of 15 matched pairs of clinical specimens containing OSCC tumors and the surrounding noncancerous mucosa tissues, LncHIFCAR was substantially up-regulated in the tumor samples with a >2 fold overexpression in 8 out of the 15 (53%) samples (FIG. 1e). Notably, as OSCC is the most common type of head and neck squamous cell carcinoma (HNSCC), we also surveyed the RNA sequencing data of The Cancer Genome Atlas (TCGA) HNSCC study using cBioPortal platform, and found a significantly up-regulated LncHIFCAR expression in HNSCC tumor samples. To assess the clinical significance of LncHIFCAR in OSCC, we next examined the relationship between LncHIFCAR expression level and the clinicopathological characteristics of 42 OSCC samples. High LncHIFCAR expression was significantly associated with age (P=0.037, Fisher's exact test) and advanced tumor grade (P=0.01, Fisher's exact test), while no significant relationship with any other clinicopathological characteristics was observed. Kaplan-Meier survival analysis was then performed to compare the outcomes of patients dichotomized by LncHIFCAR expression. Patients with high LncHIFCAR expression level had a significantly worse overall survival (OS, P=0.021, log-rank test; FIG. 1f) and recurrence-free survival (RFS, P=0.004, log-rank test; FIG. 1g) than those with low LncHIFCAR expression. Patients with high LncHIFCAR expression level had a significantly worse uterine corpus endometrial carcinoma, glioblastoma and kidney renal cell carcinoma overall survival (FIGS. 1h, I and j) than those with low LncHIFCAR expression. Moreover, similar to the prognostic values of lymph node metastasis (N0 vs. N1-3) and tumor differentiation (G1 vs. G2+G3), the univariate cox regression analysis indicated high LncHIFCAR expression as another strong prognostic predictor for poor OS (hazard ratio [HR]=2.701, P=0.037) and RFS (HR=3.686, P=0.002, Table 1). To further verify the robustness value of LncHIFCAR expression, multivariate analysis was performed to determine risk assessment related to OS and RFS. Most remarkably, high expression of LncHIFCAR was identified as an independent prognostic factor for RFS (HR=3.5, P=0.012, multivariate analysis; Table 1). Together, our data revealed that LncHIFCAR is overexpressed in OSCC/HNSCC, and that the level of LncHIFCAR could serve as an independent prognostic indicator of clinical outcomes in OSCC patients.

TABLE 1
Univariate and multivariate Cox regression analyses of overall survival and recurrence
free survival in OSCC patients

| | Univariate analysis | | Multivariate analysis | |
|---|---|---|---|---|
| Covariates | HR (95% CI) | P-value | HR (95% CI) | P-value |

Overall survival

| | | | | |
|---|---|---|---|---|
| Age (≤50 vs. >50) | 2.299 (0.872-6.062) | 0.092 | 1.860 (0.580-5.965) | 0.296 |
| T status (≤4 cm vs. >4 cm) | 2.820 (1.137-6.991) | 0.025* | 2.417 (0.584-9.991) | 0.222 |
| Stage (I + II vs. III + IV) | 3.221 (1.156-8.979) | 0.025* | 1.045 (0.164-6.636) | 0.962 |
| Tumor differentiation (G1 vs. G2 + G3) | 2.556 (1.034-6.319) | 0.042* | 1.547 (0.517-4.628) | 0.434 |
| Lymph node metastasis (N0 vs. N1-3) | 3.222 (1.259-8.239) | 0.014* | 3.193 (0.845-12.058) | 0.086 |
| LncHIFCAR expression level (high vs. | 2.701 (1.058-6.896) | 0.037* | 2.239 (0.719-6.966) | 0.163 |
| low) | | | | |

Recurrence-free survival

| | | | | |
|---|---|---|---|---|
| Age (≤50 vs. >50) | 1.575 (0.713-3.479) | 0.261 | 0.967 (0.315-2.967) | 0.954 |
| T status (≤4 cm vs. >4 cm) | 1.569 (0.697-3.526) | 0.276 | 1.827 (0.457-7.299) | 0.394 |
| Stage (I + II vs. III + IV) | 1.986 (0.897-4.394) | 0.090 | 0.602 (0.083-4.339) | 0.615 |
| Tumor differentiation (G1 vs. G2 + G3) | 2.709 (1.242-5.909) | 0.012* | 1.453 (0.608-3.469) | 0.401 |
| Lymph node metastasis (N0 vs. N1-3) | 3.043 (1.374-6.735) | 0.006** | 4.147 (0.869-19.770) | 0.074 |
| LncHIFCAR expression level (high vs. | 3.686 (1.616-8.409) | 0.002** | 3.500 (1.317-9.302) | 0.012* |
| low) | | | | |

CI, confidence interval;
HR, hazard ratio;
G1, well-differentiated;
G2, moderately differentiated;
G3, poorly differentiated;
statistically significant (*P < 0.05, **P < 0.01).

### Example 3 LncHIFCAR Contributes to Cancer Progression

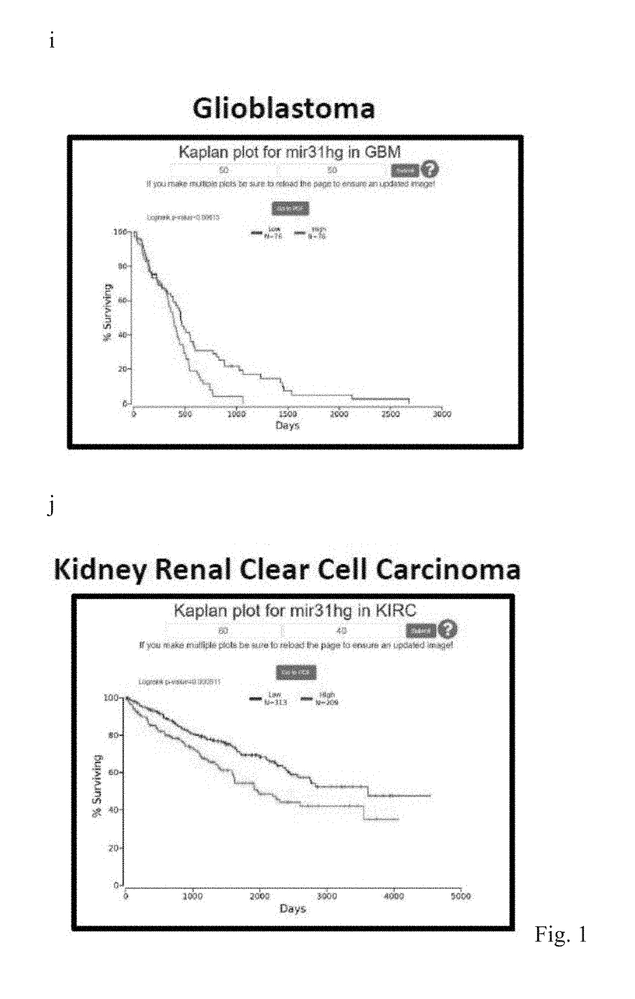

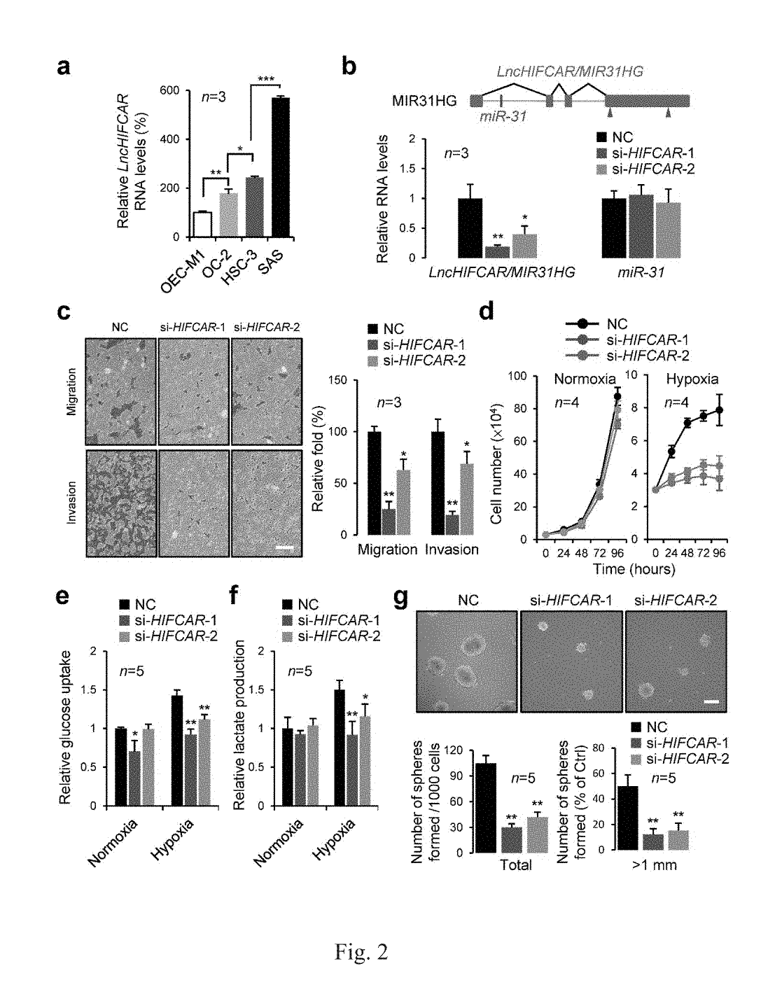

To evaluate the possible role of LncHIFCAR in oral cancer, we first analyzed the level of LncHIFCAR in a panel of OSCC cell lines, OECM1, OC-2, HSC-3 and SAS. Compared to OEC-M1 and OC-2 cells established from primary OSCC tumors that exhibit minimal invasion capacity³¹, the highly invasive HSC-3 and SAS cells expressed higher LncHIFCAR level (FIG. 2a), indicating a possible association of LncHIFCAR with the invasion ability of OSCC cell lines. Similar to HeLa cell, LncHIFCAR expression is also substantially induced in SAS cells upon chemical-induced pseudohypoxia or physical hypoxia in a dose- and time-dependent manner.

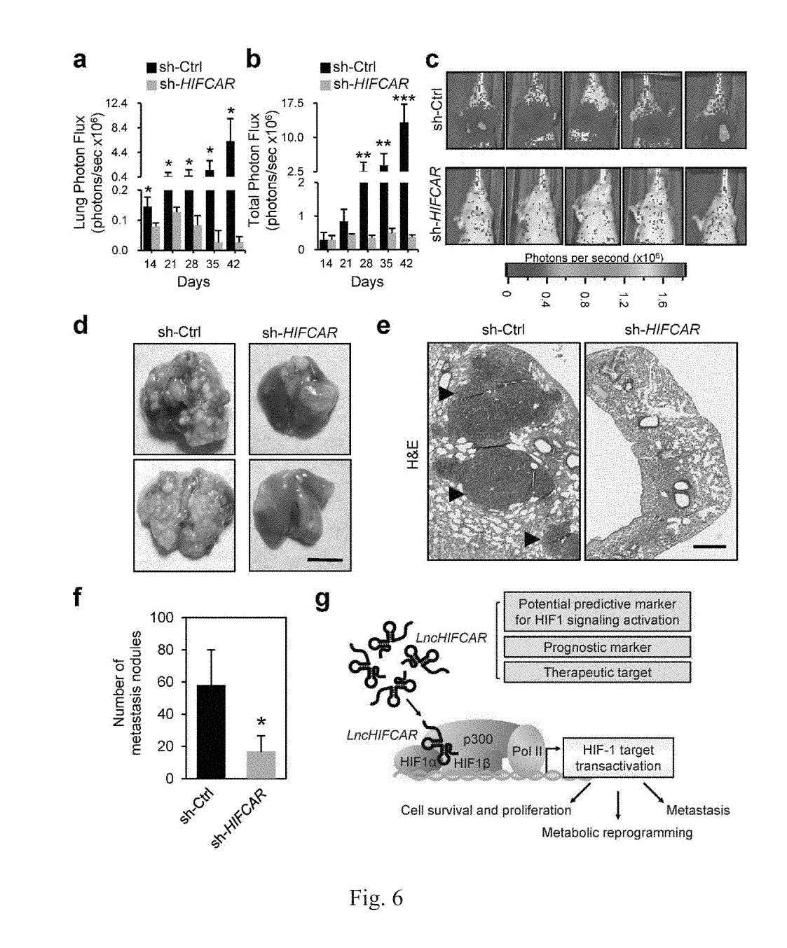

Cellular response to hypoxia is implicated in many critical aspects of cancer progression, including invasion, metastasis, stem properties maintenance and metabolism reprogramming. To further characterize the biological significance of LncHIFCAR in oral tumorigenesis, we knocked down this lncRNA with two independent small interfering RNAs (siRNAs). In addition, we used shRNA expressing-plasmids to generate stable LncHIFCAR knockdown clones (sh-HIFCAR) and vector controls (sh-Ctrl) in SAS cells, and examined the hypoxia-associated phenotypes in these cells. Notably, although miR-31 is located within the first intron of LncHIFCAR and shares the same transcription promoter, the LncHIFCAR knockdown shRNA/siRNA was designed to target the exon region, and thus only specifically downregulated LncHIFCAR but not miR-31, as determined by quantitative PCR (FIG. 2b and Supplementary FIG. 6a). As depicted in FIG. 2c and Supplementary FIG. 6b, the transwell assays showed that LncHIFCAR-knockdown resulted in a significantly impaired migration and invasion ability of SAS cells under hypoxia, echoing the positive correlation between LncHIFCAR levels and the invasion ability of different OSCC cell lines (FIG. 2a). Consistent with previous report that knockdown of LncHIFCAR impeded the growth of breast cancer cell, the LncHIFCAR-knockdown oral cancer cells also grew more slowly than the vector control (FIG. 2d) whereas LncHIFCAR-overexpression promoted the cell growth of SAS and 293T cells under normoxic and hypoxic conditions. Notably, the differences in cell proliferation became markedly more profound under hypoxic conditions (2.1 fold, FIG. 2d, right panel) than under normoxic conditions (1.2 fold, FIG. 2d, left panel), revealing a crucial role of LncHIFCAR in hypoxic cell growth. In addition, with normalization to cell number, we detected a significant reduction of hypoxia-induced glucose uptake (FIG. 2e) and lactate production (FIG. 2f) in the LncHIFCAR-knockdown SAS cells, suggesting that LncHIFCAR is essential for glycolysis in hypoxia and confers a proliferation advantage to hypoxic oral cancer cells. We then tested anchorage-independent sphere formation and found that, compared to the control cells, LncHIFCAR knockdown cells exhibited significantly weaker tumor sphere-forming ability (FIG. 2g). Together, these data suggest that LncHIFCAR acts as a critical mediator in hypoxia-associated tumorigenesis including migration, invasion, hypoxic cell growth, metabolic regulation, and the sphere-forming ability in OSCC cells.

### Example 4 LncHIFCAR Activates HIF-1 Transcriptional Network

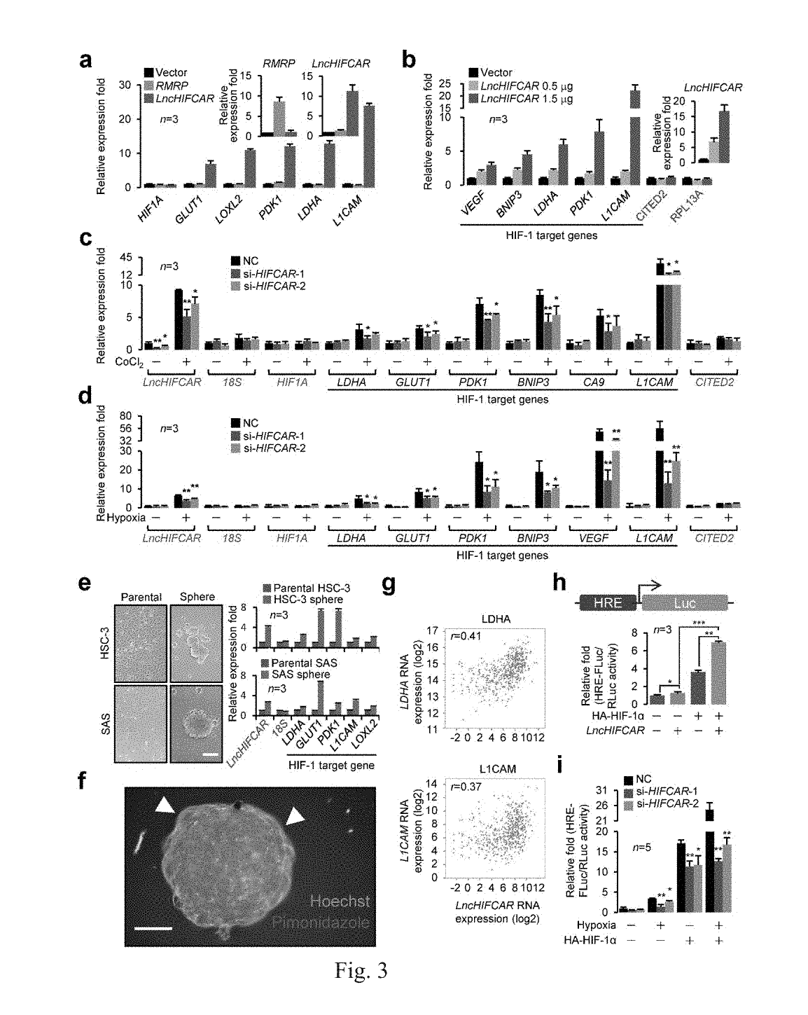

HIF-1, consisting of HIF-1α and HIF-1β subunits, is the primary player driving cellular response to hypoxia by activating the expression of target genes involved in critical steps of phenotype changes in cancer progression, including angiogenesis (VEGF, vascular endothelial growth factor), mitochondrial function (BNIP3, BCL2/Adenovirus E1B 19 kDa interacting protein 3), metabolism reprogramming (GLUT1/SLC2A1, glucose transporter 1; LDHA,lactate dehydrogenase A; CA9, carbonic anhydrase 9; PDK1, pyruvate dehydrogenase kinase isozyme 1), invasion (LICAM, L1 cell adhesion molecule) and metastasis (LOXL2, lysyl oxidase homolog 2). Given its role in the hypoxia-associated cancer phenotypes, we next investigated the impact of LncHIFCAR on the expression of HIF-1 target genes. Strikingly, overexpression of LncHIFCAR but not RMRP, another hypoxia inducible lncRNA, induced the activation of HIF-1 target genes, known as pseudohypoxia, without a significant change of HIF1A mRNA in HeLa cells under normoxic condition (FIG. 3a). Furthermore, induction of the pseudohypoxia signature showed a positive correlation with the levels of LncHIFCAR (FIG. 3b), and appeared to be HIF-1α specific, as the expression of non HIF-1 target RPL13A (ribosomal protein L13a) and HIF-2 target CITED2 (CBP/p300-interacting transactivator 2) remained unchanged upon LncHIFCAR overexpression (FIG. 3b). Reciprocally, knockdown of LncHIFCAR significantly attenuated the activation of HIF-1 specific target genes in chemical-induced pseudo-hypoxic HeLa cells (FIG. 3c), as well as in the SAS cells under physical hypoxic condition (FIG. 3d), indicating that LncHIFCAR plays a central role in the activation of HIF-1 target genes.

Remarkably, transcriptional profiling of the tumor spheres suggested that the LncHIFCAR-mediated HIF-1 activation may functionally contribute to the sphere-forming ability in OSCC cells. When we examined the expression of LncHIFCAR and the HIF-1 target genes in HSC-3 and SAS parental and their sphere-forming cells, profound up-regulation of LncHIFCAR levels accompanied with HIF-1 target gene induction was detected in the sphere-forming cells (FIG. 3e). Our findings of LncHIFCARs participation in the anchor-independent sphere formation (FIG. 2g), combined with LncHIFCAR-dependent HIF-1 target activation (FIG. 3c,d) suggest LncHIFCAR may facilitate cellular adaption to the developing hypoxia at the core of the spheres. Using the immmunohistochemical hypoxia marker pimonidazole, we observed a hypoxic gradient in the spheres (FIG. 3f). Florescence staining with propidium iodide (PI) identifies necrotic cells in these hypoxic core region of spheres. PI counterstaining of spheres with comparable size showed that knockdown of LncHIFCAR induced more necrotic cell death, suggesting that LncHIFCAR knockdown cells were unable to adapt to the developing hypoxia at the core of the spheres. Collectively, these results underscore a crucial role of LncHIFCAR in the activation of HIF-1 target genes, hypoxia adaption and sphere formation that may further contribute to tumor development.

To further validate the clinical relevance of LncHIFCAR with HIF-1 targets expression, we queried RNA sequencing and mRNA microarray data in published datasets from a variety of cancer studies. Congruent with our findings, HIF-1 target genes LDHA (Pearson's correlation r=0.41) and L1CAM (r=0.37) showed a positive correlation with the expression of LncHIFCAR in the TCGA HNSCC provisional cohort (n=522; FIG. 3g), while no significant correlation with the HIF1A mRNA levels were observed (r=0.2). Similarly, the mRNA of other HIF-1 targets such as LOXL2, PGK1, VEGFA and GLUT1 were also significantly elevated in human OSCC²⁵, invasive ductal breast carcinoma, colon and rectal adenocarcinoma that highly express LncHIFCAR, but not HIF1A mRNA (Table 2). On the other hand, the expression of HIF-2 target CITED2 was not significantly altered in the above-cancer tissues (Table 2), revealing a specific correlation of LncHIFCAR with the HIF-1 transcriptional network.

TABLE 2
Expression levels of LncHIFCAR and HIF-1α target genes in normal versus cancer
obtained from the Oncomine microarray datasets

| | Expression fold change | | | |
|---|---|---|---|---|
| | Peng Head-Neck | Zhao Breast | TCGA Colorectal | TCGA Colorectal |
| | Oral Cavity Squamous | Invasive Ductal Breast | Colon | Rectal |
| | Cell Carcinoma (57) | Carcinoma (40) | Adenocarcinoma (101) | Adenocarcinoma (60) |
| | vs. | vs. | vs. | vs. |
| RNA | Normal (22) | Normal (3) | Normal (19) | Normal (19) |
| LncHIFCAR | 4.484*** | 1.693** | 1.916*** | 1.762*** |
| LOXL2 | 1.977*** | 1.866*** | 2.437*** | 2.33*** |
| PGK1 | 1.362*** | 1.889*** | 1.982*** | 2.216*** |
| VEGFA | 1.164* | 2.598*** | 1.054*** | 1.095*** |
| GLUT1 | 1.135* | 2.8*** | 2.017*** | 2.282*** |
| LDHA | 1.414*** | 1.467** | 1.176* | NS |
| HIF1A | 1.188* | NS | NS | NS |
| CITED2 | NS | NS | NS | NS |
| (HIF-2 target) | | | | |

Statistically significant (t-test, *P < 0.05, **P < 0.01, ***P < 0.001);
NS, no statistically significant difference (t-test, P > 0.05)

We next determined whether LncHIFCAR functions through a direct effect on HIF-1 transactivation potency. A HIF-1α reporter plasmid (HRE-FLuc) containing three HREs and firefly luciferase coding sequences was used for the promoter-activity assay. Ectopic expression of HIF-1α (3.62 fold) or LncHIFCAR (1.26 fold) alone increased HIF-1 transcriptional activity, while co-expression of HIF-1α and LncHIFCAR synergistically enhanced the promoter activity (6.94 fold; FIG. 3h). In a reciprocal experiment, LncHIFCAR-silencing led to a significant reduction in hypoxia-stimulated or ectopic HIF-1α-induced HIF-1 transcriptional activation (FIG. 3i). Taken together, these data suggest that LncHIFCAR augments the transcriptional activity of HIF-1α, and serves as a critical regulator of HIF-1 transcriptional network, which enhances the anchorage-independent growth and provides a growth advantage for tumor cells for hypoxia adaptation.

### Example 5 LncHIFCAR Acts as a HIF-1α Co-Activator

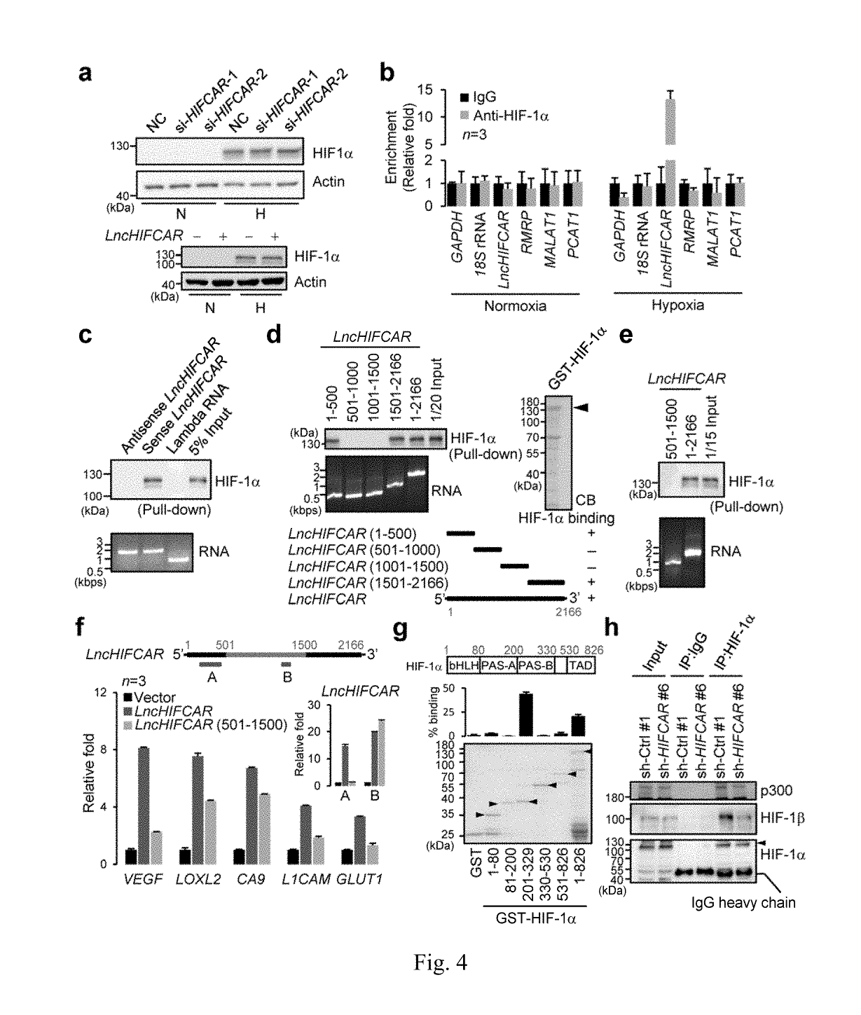

The major mechanism of HIF-1 activation in hypoxia is attributed to the stabilization of HIF-1α protein. We found that neither overexpression nor knockdown of LncHIFCAR had a significant impact on the protein level of HIF-1α either under normoxic, chemical induced pseudo-hypoxic or physical hypoxic conditions (FIG. 4a). Additionally, western blotting analysis of nuclear and cytosolic fractions revealed no detectable changes in the hypoxia-induced HIF-1α nuclear accumulation in LncHIFCAR-knockdown cells compared with control cells. Meanwhile, there is no noticeable difference in the subcellular localization of the LncHIFCAR lncRNA in normoxic and hypoxic conditions. These results suggest a mechanism for LncHIFCAR-mediated HIF-1 activation independent of protein stabilization and translocation of HIF-1α.

Under normoxia conditions, the expression of HIF-1α proteins is generally low. We asked whether this low HIF-1α expression is required for LncHIFCAR-induced pseudohypoxia signature and HIF-1 transactivation as seen in FIGS. 3a, 3b and 3h. We found that they both were reverted by HIF-1α knockdown, indicating the critical role of HIF-1α in LncHIFCAR-mediated HIF-1 target gene regulation. Therefore, we next examined the physical interaction of LncHIFCAR and HIF-1α. Using nuclear extracts of SAS cells, RNA immunoprecipitation (RIP) assay showed a robust and specific enrichment of LncHIFCAR co-precipitated within the HIF-1α immunocomplex in cells under hypoxia, while no enrichment was detected for other RNA molecules such as GAPDH mRNA, 18S rRNA, a comparable sized lncRNA PCAT1, and hypoxia-inducible RMRP and MALAT1 lncRNAs (FIG. 4b). The physical interaction between LncHIFCAR and HIF-1α was confirmed by RNA pull-down assay using hypoxic cell nuclear extracts incubating with in vitro transcribed biotinylated LncHIFCAR (FIG. 4c). With purified recombinant HIF-1α protein in the in vitro binding assay, we further validated that LncHIFCAR associated with HIF-1α through direct binding (FIG. 4d).

To delineate the structural determinants for the association between LncHIFCAR and HIF-1α, RNA pull-down assays were performed with a series of LncHIFCAR truncated fragments. Both the 5′-terminal (nucleotides 1-500) and 3′-terminal (nucleotides 1500-2166) regions were found to be associated with HIF-1α (FIG. 4d). As expected, the HIF-1α association was abolished when using a truncated LncHIFCAR (nucleotides 501-1500) that lacked the two binding regions (FIG. 4e), confirming that the critical HIF-1α-interaction regions in LncHIFCAR reside at the 5′-terminal (nucleotides 1-500) and 3′-terminal (nucleotides 1500-2166) sections. To further explore functional relevance of the LncHIFCAR/HIF-1α interaction, the full-length and deletion mutant LncHIFCAR (501-1500) were overexpressed in SAS cells. As shown in FIG. 4f, relative to full-length LncHIFCAR, the ability of the HIF-1α binding-deficient mutant LncHIFCAR (501-1500) to induce HIF-1 target genes and HIF-1 responsive luciferase reporter was severely impaired. This result suggests that the HIF-1α-binding regions functionally contribute to the LncHIFCAR-mediated HIF-1 target induction. We next mapped HIF-1α domain required for LncHIFCAR binding by generating HIF-1α bHLH (basic helix-loop-helix; amino acids 1-80), PAS-A (Per-ARNT-Sim-A; amino acids 81-200), PAS-B (Per-ARNT-Sim-B; amino acids 201-329), and TAD (transactivation domain; amino acids 191-445) deletion mutants (FIG. 4g, bottom panel). In vitro pull-down assays revealed that LncHIFCAR strongly bound to the PAS-B domain of HIF-1α (FIG. 4g). As this domain is responsible for functional dimerization, we then conducted immunoprecipitation of HIF-1α with hypoxic cell lysate to investigate the effect of LncHIFCAR on HIF1 complex formation. Knockdown of LncHIFCAR reduced the interaction between HIF-1α and both HIF-113 and p300 (FIG. 4h), indicating that LncHIFCAR could facilitate the recruitment of HIF-1 complex. Collectively, the data presented in FIG. 3 and FIG. 4 indicates that LncHIFCAR functions as a transcriptional co-activator of HIF-1α. Given that the HIF-1α level in many tumor cells can remain elevated under normoxic conditions, LncHIFCAR may induce pseudohypoxia signature by promoting HIF-1 complex formation under normoxia.

### Example 6 LncHIFCAR Enhances HIF-1 Complex Binding to the Target Loci

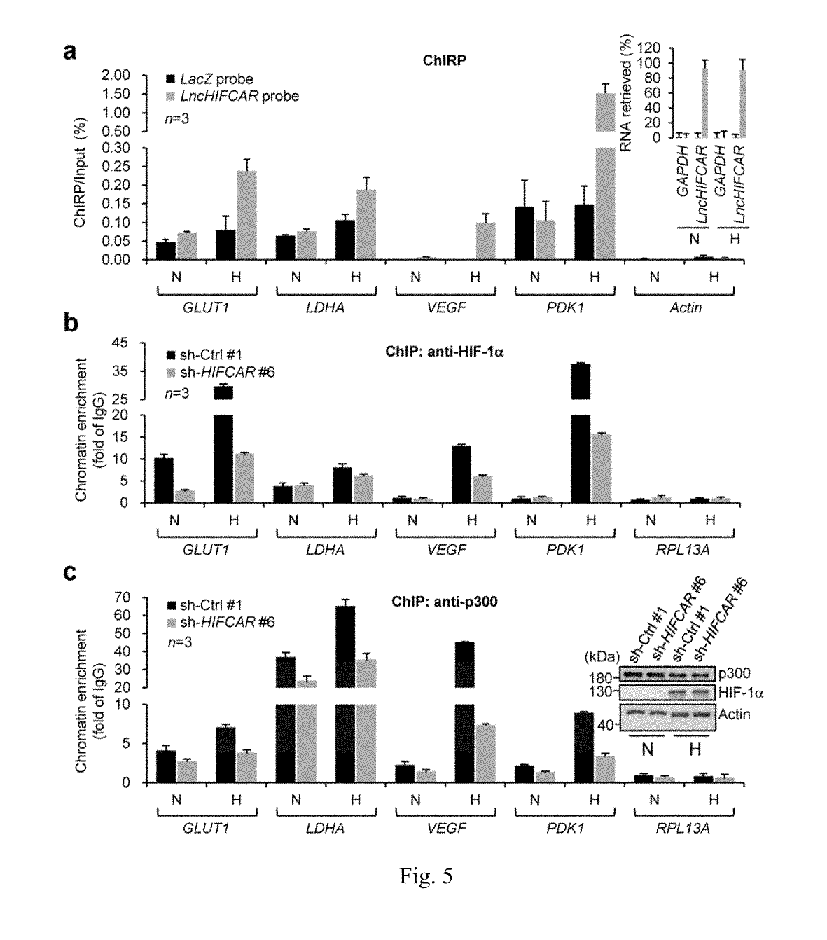

To verify whether LncHIFCAR directly acts on the target chromatins, we analyzed the loci of LncHIFCAR-dependent HIF-1 target genes as identified in FIG. 3d by employing chromatin isolation by RNA purification (ChIRP). A set of probes complementary to LncHIFCAR was used to pull down the endogenous LncHIFCAR from normoxic or hypoxic SAS cells, and the promoter regions of known HIF-1α binding sites were amplified and quantified by qPCR. In concordance with the notion that LncHIFCAR functions as a HIF-1α co-activator, the ChIRP analysis showed that LncHIFCAR is recruited to a subset of the HIF-1 target promoters in hypoxic cells, suggesting overlapping chromatin occupancy of LncHIFCAR and HIF-1α under hypoxia (FIG. 5a). Since hypoxia-induced target activation is mediated by HIF-1 transcriptional complex composed of HIF-1 heterodimer and the transcription cofactor p300/CBP, we next examined whether the chromatin binding of LncHIFCAR is involved in the recruitment of HIF-1α and the cofactor upon hypoxia. Significantly, the ChIP assays using anti-HIF-1α and anti-p300 antibodies showed that these transcriptional factors were enriched on the target promoters in hypoxic cells, while knockdown of LncHIFCAR significantly reduced this chromatin enrichment (FIG. 5b,c). These results suggest that under hypoxia, LncHIFCAR forms a complex with HIF-1α, and enhances the chromatin recruitment of HIF-1α and p300 cofactor, thereby promoting the transcriptional reprogramming essential in multiple oncogenic pathways.

### Example 7 LncHIFCAR Promotes Metastatic Cascade

With our evidence of LncHIFCAR in HIF-1 activation and hypoxia-associated cancer phenotypes, the biological significance of LncHIFCAR in oral cancer progression in vivo was examined using mouse xenograft model. The vector control and LncHIFCAR knockdown SAS cells were genetically modified to express firefly luciferase, and subsequently injected intravenously into nude mice (Day 0). Following tail vein inoculation, the bioluminescence intensity of the lung region was measured weekly up to 6 weeks. While the vector control cells exhibited strong colonization to the lung with a timely progression, LncHIFCAR-knockdown cells showed a drastically reduced ability to colonize in lung (FIG. 6a,b,c). Verified by histology, a significantly increased incidence of pulmonary metastasis was observed in the mice bearing vector control cells, whereas only rare tumor foci were found in the lungs of mice carrying LncHIFCAR knockdown cells (FIG. 6d,e,f). Notably, the difference in tumor foci size and occurrence cannot be fully accounted for by cell proliferation rate caused by LncHIFCAR modulation under normoxia. As judged from the time-lapsed image, control vector-expressing cells colonized to the lung 14 days after tail vein injection. By contrast, the LncHIFCAR knockdown xenografts showed significant less lung colonization at the same time point when no apparent change of the injected cell density was observed compared to the control group (Day 14, FIG. 6a,b). Given the critical role of hypoxic signaling in the formation of a premetastatic niche essential for tumor colonization at the distant site, we propose that LncHIFCAR-mediated difference in colonization ability is a combined effect of enhanced HIF-1 signaling activation and increased hypoxic tumor cell growth advantage conferred by this lncRNA. Altogether, these results suggest that LncHIFCAR enhanced the metastatic spread of SAS oral cancer cells in vivo, further substantiating its oncogenic potential in OSCC progression.

What is claimed is:

1. A method of diagnosing whether a subject has, or is at risk for a cancer, a metastatic cancer or a primary cancer, comprising:
a) isolating a LncHIFCAR transcript in a biological sample from the subject;
b) measuring a test level of the isolated LncHIFCAR transcript;
c) comparing the test level to a control level of the LncHIFCAR transcript; and
d) determining a subject as having the cancer, metastatic cancer or primary cancer when the test level is higher than the control level.

2. The method of claim 1, wherein the diagnosis includes a diagnosis in various stages of a cancer.

3. The method of claim 1, wherein the diagnosis is in early stage, invasion stage and metastatic stage of a cancer.

4. The method of claim 1, wherein the cancer is an oral cancer (such as an oral squamous cell carcinoma (OSCC)) or a hypoxia-mediated oral cancer, brain cancer (such as glioblastoma), kidney cancer (such as kidney renal clear cell carcinoma) or a hypoxia-mediated brain cancer, colorectal cancer or a hypoxia-mediated colorectal cancer, or uterine cancer (such as uterine corpus endometrial carcinoma) or a hypoxia-mediated uterine cancer.

5. The method of claim 1, wherein the LncHIFCAR level is detected and quantitated by microarray analysis, polymerase chain reaction (PCR), reverse transcriptase polymerase chain reaction (RT-PCR), Northern blot, serial analysis of gene expression (SAGE), immunoassay, mass spectrometry or a sequencing-based method.

6. The method of claim 1, wherein the biological sample is a sample of tissue or fluid isolated from a subject.

7. The method of claim 1, the method further includes a step of administering a siRNA silencing LncHIFCAR to treat the cancer.

8. The method of claim 7, wherein the siRNA comprises a sequence selected from the group consisting of SEQ ID NO:1, 2, 3 and 4.

9. The method of claim 1, wherein the gene expressing LncHIFCAR is MIR31HG whose sequence is disclosed in NCBI Reference Sequence: NR_027054.1, NR_027054.2, NR_152877.1, NR_152878.1 or NR_152879.1.

10. A method of determining a prognosis, recurrence-free survival or overall survival of a subject having, or suspected of a cancer, a metastatic cancer or a primary cancer, comprising: a) isolating a LncHIFCAR transcript in a biological sample from the subject; b) measuring a test level of the isolated LncHIFCAR transcript; c) comparing the test level to a control level of the LncHIFCAR transcript; and d) determining a subject as having a poor prognosis, poor recurrence-free survival or poor overall survival when the test level is higher than the control level.

11. The method of claim 10, wherein the determination of a prognosis can be used as an independent prognostic factor.

12. The method of claim 10, wherein the biological sample is a sample of tissue or fluid isolated from a subject.

13. The method of claim 10, wherein the LncHIFCAR level is detected and quantitated by microarray analysis, polymerase chain reaction (PCR), reverse transcriptase polymerase chain reaction (RT-PCR), Northern blot, serial analysis of gene expression (SAGE), immunoassay, mass spectrometry or a sequencing-based method.

14. The method of claim 10, wherein the gene expressing LncHIFCAR is MIR31HG whose sequence is disclosed in NCBI Reference Sequence: NR_027054.1, NR_027054.2, NR_152877.1, NR_152878.1 or NR_152879.1.

15. The method of claim 10, wherein the cancer is an oral cancer (such as an oral squamous cell carcinoma (OSCC)) or a hypoxia-mediated oral cancer, brain cancer (such as glioblastoma), kidney cancer (such as kidney renal clear cell carcinoma) or a hypoxia-mediated brain cancer, colorectal cancer or a hypoxia-mediated colorectal cancer, or uterine cancer (such as uterine corpus endometrial carcinoma) or a hypoxia-mediated uterine cancer.

16. The method of claim 1, the method further includes a step of administering a siRNA silencing LncHIFCAR to treat the cancer.

17. The method of claim 16, wherein the siRNA comprises a sequence selected from the group consisting of SEQ ID NO:1, 2, 3 and 4.

18. A kit for predicting a risk for developing a cancer, a metastatic cancer or a primary cancer or a prognosis, recurrence-free survival or overall survival of a subject, comprising reagents for determining a level of the LncHIFCAR in the sample.

19. A method of treating a cancer, a metastatic cancer and/or a primary cancer in a subject comprising administering to the subject an effective amount of a therapeutic agent that blocks an expression or overexpression of MIR31HG gene or a physiological action of a LncHIFCAR transcript.

20. The method of claim 19, wherein the therapeutic agent is an antisense oligonucleotide, an antisense RNA, a small molecular inhibitor, an antisense cDNA, RNA, siRNA, esiRNA, shRNA, miRNA, decoy, RNA aptamer, RNA/DNA demethylating agent and RNA/DNA-binding protein/peptide or a compound to inhibit one or more physiological actions affected by LncHIFCAR.

21. The method of claim 20, wherein the therapeutic agent is a siRNA comprising a sequence of SEQ ID NO:1, 2, 3 or 4.


SEQUENCE LISTING (4 sequences)

SEQ ID NO: 1 - DNA, 21 bp, artificial sequence
  synthetic polynucleotide
ggguuucuguauucaguuatt

SEQ ID NO: 2 - DNA, 21 bp, artificial sequence
  synthetic polynucleotide
uaacugaauacagaaaccctt

SEQ ID NO: 3 - DNA, 21 bp, artificial sequence
  synthetic polynucleotide
ccagcugcugaugacguaatt

SEQ ID NO: 4 - DNA, 21 bp, artificial sequence
  synthetic polynucleotide
uuacgucaucagcagcuggtt